(19) United States
(12) Patent Application Publication
(10) Pub. No.: US 2021/0270742 A1
(43) Pub. Date: Sep. 2, 2021

(54) PEAK-PRESERVING AND ENHANCING BASELINE CORRECTION METHODS FOR RAMAN SPECTROSCOPY

(71) Applicant: Virginia Tech Intellectual Properties, Inc., Blacksburg, VA (US)

(72) Inventors: Ryan Senger, Richmond, VA (US); Pang Du, Blacksburg, VA (US); John L. Robertson, Floyd, VA (US); Yunnan Xu, Blacksburg, VA (US)

(21) Appl. No.: 17/188,737

(22) Filed: Mar. 1, 2021

Related U.S. Application Data
(60) Provisional application No. 62/983,045, filed on Feb. 28, 2020.

(51) Int. Cl. G01N 21/65; G01N 33/493; A61B 5/00

(52) U.S. Cl. CPC G01N 21/65; G01N 33/493; G01N 2201/127; A61B 5/7264; A61B 5/0075

(57) ABSTRACT

Baseline correction in Raman spectroscopy is a procedure that eliminates/reduces the background signals generated by residual Rayleigh scattering or fluorescence. Provided is a novel baseline correction procedure called the Iterative Smoothing-splines with Root Error Adjustment (ISREA) that has three distinct advantages. First, ISREA uses smoothing splines, which are more flexible than polynomials and capable of capturing complicated trends over the whole spectral domain, to estimate the baseline. Second, ISREA mimics the asymmetric square root loss and removes the need of a threshold. Finally, ISREA avoids the direct optimization of a non-convex loss function by iteratively updating prediction errors and refitting baselines. Through extensive numerical experiments on a wide variety of spectra including simulated spectra, mineral spectra, and dialysate spectra, the present inventors show that ISREA is simple, fast, and can yield consistent and accurate baselines that preserve all the meaningful Raman peaks.

### CROSS-REFERENCE TO RELATED APPLICATIONS

This application relies on the disclosure of and claims priority to and the benefit of the filing date of U.S. Provisional Application No. 62/983,045 filed Feb. 28, 2020, which is hereby incorporated by reference herein in its entirety.

### STATEMENT OF GOVERNMENT INTEREST

This invention was supported by the U.S. National Science Foundation under Contract Nos. DMS-1620945 and DMS-1916174. The U.S. government has certain rights in this invention.

### BACKGROUND OF THE INVENTION

### Field of the Invention

The present invention relates to the field of disease detection and characterization. More particularly, the present invention relates to methods of detecting or characterizing disease and/or monitoring disease therapy using Raman spectroscopy of patient samples.

### Description of Related Art

Raman spectroscopy is an established tool used for both qualitative and quantitative analyses of molecular composition of macro- and nano-materials and biological systems (A. Kudelski. “Analytical applications of Raman spectroscopy”. Talanta. 2008. 76(1): 1-8). More recently, advances in instrumentation have improved sensitivity greatly, reduced interference from fluorescence and environmental sources, and have led to spectrometers that are relatively inexpensive and have a small footprint (R. S. Das, Y. K. Agrawal. “Raman spectroscopy: Recent advancements, techniques and applications”. Vib. Spectrosc. 2011. 57(2): 163-176; N. Wang, H. Cao, L. Wang, F. Ren, Q. Zeng, X. Xu, et al. “Recent advances in spontaneous Raman spectroscopic imaging: Instrumentation and applications”. Curr. Med. Chem. 2019. 26: 1). It has also found applications in a variety of medical studies, such as disease diagnosis and monitoring efficacy and progress of therapy (J. Depciuch, E. Kaznowska, I. Zawlik, R. Wojnarowska, M. Cholewa, P. Heraud, et al. “Application of Raman Spectroscopy and Infrared Spectroscopy in the Identification of Breast Cancer”. Appl. Spectrosc. 2016. 70(2): 251-263; A. K. Fisher, W. F. Carswell, A. I. M. Athamneh, M. C. Sullivan, J. L. Robertson, D. R. Bevan, et al. “The Rametrix™ LITE Toolbox v1.0 for MATHLAB®”. J. Raman Spectrosc. 2018. 49(5): 885-896; R. S. Senger, V. Kavuru, M. Sullivan, A. Gouldin, S. Lundgren, K. Merrifield, et al. “Spectral characteristics of urine specimens from healthy human volunteers analyzed using Raman chemometric urinalysis (Rametrix)”. PLoS One. 2019. 14(9): e0222115; R. S. Senger, J. L. Robertson. “The Rametrix™ PRO Toolbox v1.0 for MATLAB®”. PeerJ. 2020. 8: e8179; R. S. Senger, M. Sullivan, A. Gouldin, S. Lundgren, K. Merrifield, C. Steen, et al. “Spectral characteristics of urine from patients with end-stage kidney disease analyzed using Raman Chemometric Urinalysis (Rametrix)”. PLoS One. 2020. 15(1): e0227281).

In Raman spectrum generation, a background signal generated by fluorescence or Rayleigh scattering can heavily interfere with accurate analysis of the underlying Raman spectrum. This background signal, commonly known as the baseline, often appears as a smooth curve in the raw spectrum. Therefore, one critical step in Raman spectroscopy is to perform a baseline correction that involves estimating the baseline by a smooth function and then removing it from the raw spectrum by subtraction (R. Gautam, S. Vanga, F. Ariese, S. Umapathy. “Review of multidimensional data processing approaches for Raman and infrared spectroscopy”. EPJ Techn. Instrum. 2015. 2(1): 8).

Numerous baseline correction methods have been proposed over the years. Almost all baseline correction methods implement an algorithm that employs a smoother to capture the smooth trend and a loss function to adjust the fitting. Commonly used smoothers include first- and second-order differentiation, Fourier transformation (P. A. Mosier-Boss, S. H. Lieberman, R. Newbery. “Fluorescence rejection in Raman spectroscopy by shifted-spectra, edge detection, and FFT filtering techniques”. Appl. Spectrosc. 1995. 49: 630-638; J. Zhao, H. Lui, D. I. McLean, H. Zeng. “Automated autofluorescence background subtraction algorithm for biomedical Raman spectroscopy”. Appl. Spectrosc. 2007. 61(11): 1225-1232), polynomial fitting (A. Mahadevan-Jansen, R. R. Richards-Kortum. “Raman spectroscopy for the detection of cancers and precancers”. J. Biomed. Opt. 1996. 1(1): 31-70; C. A. Lieber, A. Mahadevan-Jansen. “Automated Method for Subtraction of Fluorescence from Biological Raman Spectra”. Appl. Spectrosc. 2003. 57: 1363-1367), splines (C. M. Stellman, K. S. Booksh, M. L. Myrick. “Multivariate Raman Imaging of Simulated and “Real World Glass-Reinforced Composites”. Appl. Spectrosc. 1996. 50: 552-557; V. Shusterman, S. I. Shah, A. Beigel, K. P. Anderson. “Enhancing the precision of ECG baseline correction: selective filtering and removal of residual error”. Comput. Biomed. Res. 2000. 33(2): 144-160; G. Schulze, A. Jirasek, M. M. Yu, A. Lim, R. F. Turner, M. W. Blades. “Investigation of selected baseline removal techniques as candidates for automated implementation”. Appl. Spectrosc. 2005. 59(5): 545-574), and wavelets (T. T. Cai, D. Zhang, D. Ben-Amotz. “Enhanced Chemical Classification of Raman Images Using Multiresolution Wavelet Transformation”. Appl. Spectrosc. 2001. 55(9): 1124-1130; D. Chen, Z. Chen, E. Grant. “Adaptive wavelet transform suppresses background and noise for quantitative analysis by Raman spectrometry”. Anal. Bioanal. Chem. 2011.400(2): 625-634; J. Li, B. Yu, H. Fischer. “Wavelet transform based on the optimal wavelet pairs for tunable diode laser absorption spectroscopy signal processing”. Appl. Spectrosc. 2015. 69(4): 496-506). For example, Zhang and Ben-Amotz used the Savitzky-Golay second-derivative method on spectral data (D. Zhang, D. Ben-Amotz. “Enhanced Chemical Classification of Raman Images in the Presence of Strong Fluorescence Interference”. Appl. Spectrosc. OSA, 2000. 54(9): 1379-1383). Although differentiation is unbiased and efficient for fluorescence subtraction, it can severely distort shapes of Raman spectra and relies on complex fitting algorithms to reproduce conventional spectra according to Mosier-Boss and Lieberman, who applied the Fast Fourier transform (FFT) filtering technique to Raman spectra to eliminate interference due to fluorescence (P. A. Mosier-Boss et al., 1995). Fourier transform filtering depends on direct human intervention to choose its upper and lower limits in the frequency domain each time. This process is tedious and does not lend well to automated analyses. In contrast, polynomials are simple and convenient and thus popular in the biomedical field.

To preserve peak intensities of Raman spectra, they often rely on manual recognition of background points. Otherwise, the fitted baseline would include both fluorescence background and Raman peaks. Furthermore, the manual selection of background points can be time consuming, which, again, does not lend well to automated spectra processing. To avoid the human-intervention input to the selection process, iterative procedures were considered (J. Zhao et al., 2007; C. A. Lieber et al., 2003). Splines were used in baseline correction as early as the 1990s (C. M. Stellman et al., 1996). More recently, Cai et al. presented a method that combines penalized B-splines with vector transformation (Y. Cai, C. Yang, D. Xu, W. Gui. “Baseline correction for Raman spectra using penalized spline smoothing based on vector transformation”. Anal. Methods. 2018. 10(28): 3525-3533). Wavelet transformation has been another popular tool for baseline correction. For example, Cai et al. applied the multiresolution wavelet transformation (MWT) and suppressed the empirical wavelet coefficients in groups with a blockwise threshold (T. T. Cai et al., 2001). However, the major drawbacks of wavelets are their assumption of a well separated background from the rest of the signal (V. Mazet, C. Carteret, D. Brie, J. Idier, B. Humbert. “Background removal from spectra by designing and minimising a non-quadratic cost function”. Chemom. Intell. Lab. Syst. 2005. 76(2): 121-133) and the need for selecting the wavelet type and the wavelet coefficient threshold. Furthermore, wavelets may sometimes lead to a sub-optimal filter for experimental signals. A second-generation adaptive wavelet transform, which makes use of a spatial domain to generate new wavelet filters, was also developed (D. Chen et al., 2011). Yet, it is still complex and computationally expensive to implement, limiting its practical application.

Also commonly seen are model-based approaches where baseline correction involves specifying models for additive and multiplicative forms of background noises. One example is the singular value decomposition (SVD)-based method where multivariate loadings were used for background correction (J. R. Beattie, J. J. McGarvey. “Estimation of signal backgrounds on multivariate loadings improves model generation in face of complex variation in backgrounds and constituents”. J of Raman Spectrosc. 2013. 44(2): 329-338. 10.1002/jrs.4178). Another example is the extended multiplicative signal correction (EMSC), which was first proposed for the near infrared spectroscopy (H. Martens, E. Stark. “Extended multiplicative signal correction and spectral interference subtraction: new preprocessing methods for near infrared spectroscopy.” J. Pharm. Biomed. Anal. 1991. 9 8: 625-635) and later extended to Raman spectra (K. Liland, A. Kohler, N. Afseth. “Model-based pre-processing in Raman spectroscopy of biological samples”. J. Raman Spectrosc. 2016. 47(6): 643-650; M. Scholtes-Timmerman, H. Willemse-Erix, T. B. Schut, A. van Belkum, G. Puppels, K. Maquelin. “A novel approach to correct variations in Raman spectra due to photo-bleachable cellular components”. Analyst. 2009. 134(2): 387-393; P. Candeloro, E. Grande, R. Raimondo, D. D. Mascolo, F. Gentile, M. L. Coluccio, et al. “Raman database of amino acids solutions: A critical study of extended multiplicative signal correction”. Analyst. 2013. 138(24): 7331-7340). An EMSC model decomposes a raw spectrum into three components: a polynomial baseline function, a multiple of a reference spectrum, and the residual that actually contains the spectrum of interest for the scanned sample. Through an ordinary or weighted least squares estimation, it produces spectra similar to the reference spectrum. The choice of the reference spectrum thus depends on the ensuing analysis. When peak heights or related information are not required, a commonly used reference spectrum is the mean spectrum. Otherwise, a baseline-corrected reference spectrum should be used to achieve baseline correction for all spectra (N. K. Afseth, A. Kohler. “Extended multiplicative signal correction in vibrational spectroscopy, a tutorial”. Chemometrics and Intelligent Laboratory Systems. 2012. 117: 92-99. 10.1016/j.chemolab.2012.03.004).

In addition to the smoother, the choice of the loss function is also critical for baseline correction. Most existing methods rely on a symmetric loss function, generally the least square loss. This is now recognized as inappropriate for the baseline correction purpose (J. Liu, J. Sun, X. Huang, G. Li, B. Liu. “Goldindec: A Novel Algorithm for Raman Spectrum Baseline Correction”. Appl. Spectrosc. 2015. 69(7): 834-842) as it tends to produce a baseline that invades into Raman peaks. More specifically, a high peak in a raw spectrum is often expected to be made up of a Raman scattering signal represented by high peaks and a smooth baseline signal that forms the bottom of the peaks. A fitted function based on the least squares loss, however, often cuts into the peak areas instead of properly estimating the bottom of the peaks. Therefore, the heights of the peaks in the baseline-corrected spectrum would be smaller than their true values. This can create problems for ensuing quantitative analysis of Raman spectra since, according to Beer's Law, there is a proportional relationship between the height of a peak and the concentration of the molecule(s) creating it. The deficiency of the least squares loss lies in that it often pulls up the fitted curve to match up with a peak in order to minimize their squared difference.

Based on the limitations noted in these observations, several asymmetric loss functions have been proposed. These asymmetric functions share the common feature of a reduced loss for large positive deviations compared with the least squares loss. The asymmetric least squares (ALS) loss was first proposed, which is essentially a weighted least squares with second-order derivatives as the penalty term (P. H. C. Eilers. “A perfect smoother”. Anal. Chem. 2003. 75(14): 3631-3636). However, the ALS may produce artificial negative peaks on the corrected spectrum (K. Liland, T. Almøy, B. Mevik. “Optimal Choice of Baseline Correction for Multivariate Calibration of Spectra”. Appl. Spectrosc. 2010. 64: 1007-1016). Peng et al. generalized this approach for multiple spectra baseline correction taking advantage of means of the similarity among the multiple spectra (J. Peng, S. Peng, J. A, J. Jiping Wei, C. Changwen Li, J. Jie Tan. “Asymmetric least squares for multiple spectra baseline correction”. Anal. Chim. Acta. 2010. 683(1): 63-68). They assumed that the baseline stays the same or changes little for spectra of samples collected continuously over time. He et al. proposed an improved asymmetric least squares method, which adds the first-order derivative of residuals to the least squared loss to achieve smoothness and used second-order polynomials as the smoother (S. He, W. Zhang, L. Liu, Y. Huang, J. He, W. Xie, et al. “Baseline correction for Raman spectra using an improved asymmetric least squares method”. Anal. Methods. The Royal Society of Chemistry, 2014. 6(12): 4402-4407). Mazet et al. replaced the symmetric squared loss with asymmetric Huber loss and asymmetric truncated quadratic loss to suppress the effect of large positive residuals, i.e. peak areas, and estimated the baseline by a low-order polynomial (V. Mazet et al., 2005). But selections of two tuning parameters, the threshold of loss functions and the order of polynomial, can be tricky. The order of polynomial demands manual selection based on the smoothness of background. The authors suggested that splines may provide a better fitting than polynomials.

More recently, Liu et al. developed the Goldindec method using polynomials with an asymmetric Indec loss and implemented it through a half-quadratic algorithm (J. Liu et al., 2015). This method was chosen for baseline correction in the initial versions of the Raman Chemometrics (Rametrix™) LITE and PRO Toolboxes for MATLAB® (A. K. Fisher et al., 2018; R. S. Senger et al., PeerJ. 2020). Among asymmetric losses, the asymmetric Indec loss has been shown to have the best performance. However, one major drawback of the Goldindec algorithm is its use of polynomials to represent a baseline signal. Polynomials, especially low-degree polynomials, are often used to fit simple smooth functions. Due to its small number of tuning parameters (the coefficients in a polynomial), polynomials may not be sufficiently flexible to capture complicated smooth trends and can be easily distorted by a few influential points. Another drawback is that Goldindec requires the selection of a change point in the asymmetric Indec loss function. This change point is critical for the success of the algorithm since it specifies the threshold such that a positive deviation beyond it will be less penalized than in the least squares loss. However, there is no systematic way to choose this change point, although some empirical (i.e., subjective) choices are suggested and their performance may not be satisfactory in practice.

As described above, a simple polynomial or smoothing spline fitted to a raw Raman spectrum is likely to produce a baseline that moves up with peaks, while a true baseline should leave the peaks untouched and take out only the background signals. That is, after the subtraction of a well-estimated baseline, the remaining spectrum should have the peak intensities preserved in the peak areas and intensities close to zero in the non-peak areas. Therefore, the present inventors have developed a baseline correction method whose baseline estimate stays close to the true baseline with all the interesting peaks well-preserved.

### SUMMARY OF THE INVENTION

Rametrix™ brings a new method for analyzing Raman spectra. It is divided into two toolboxes: (1) Rametrix™ Signal Processing and (2) Rametrix™ Data Analysis. Novel inventions are included in Rametrix™ Signal Processing and are discussed below. The Rametrix™ Data Analysis portion includes statistical and chemometric models and approaches that have been used in other analyses available in the literature.

Rametrix™ Signal Processing and Data Analysis enables detection of the presence and/or quantification of several diseases and pathologies (such as bladder cancer, acute and/or chronic cystitis, schistosomiasis, kidney cancer, prostate cancer, prostatitis, cervical cancer, uterine cancer, ovarian cancer, cancer of the adrenal gland, Cushing's disease and Cushing's syndrome, multiple myeloma with Bence-Jones proteinuria, acute kidney injury, acute and/or chronic kidney failure, acute and/or chronic glomerulonephritis, focal and diffuse segmental glomerulosclerosis, hypertension, membranous nephropathy, membranoproliferative glomerulonephritis, hemolytic uremic syndrome, IgA nephropathy, minimal change nephropathy, congenital nephropathy, diabetes, diabetic nephropathy, protein-losing nephropathy and nephrotic syndrome, acute and/or chronic pyelonephritis, lyme disease, atypical borreliosis, myalgic encephalomyelitis/chronic fatigue syndrome, systemic mold allergy/toxicity, hemobartonellosis, SARS-CoV-1, SARS-CoV-2, and/or MERS-CoV-2) from routine Raman analysis of a sample, such as liquid urine or a dialysate. Exemplary patent applications of the inventors relating to this technology include International Patent Publication Nos. WO2015/164620 and WO2020/176663, as well as U.S. Patent Publication No. 2017/0045455 and U.S. patent application Ser. No. 17/146,301, which are each hereby incorporated by reference herein in their entireties.

Two recent developments in Rametrix™ Signal Processing have resulted in vastly improved data analysis capabilities compared to other technologies available and thus more accurate results. In particular, the inventions subtract fluorescence from a Raman spectrum and highlights regions of the spectrum where valuable spectral information reside.

The first is ISREA, a novel Raman spectrum baselining procedure (Xu, Y., Du, P., Senger, R. S., Robertson, J. L., Pirkle, J. L. (2021) ISREA: An efficient peak-preserving baseline correction algorithm for Raman spectra. Applied Spectroscopy. 75(1):34-45, which is hereby incorporated by reference in its entirety).

The second is a method for optimizing the implementation of ISREA (Carswell, W. F., Senger, R. S., & Robertson, J. L. (2021). Raman spectroscopic detection and quantification of macro- and microhematuria in human urine. Applied Spectroscopy. (In revision); see also Carswell, W. F., Grant, D., Cole, M., Guruli, G., Senger, R. S., Robertson, J. L. (2021) Detection of bladder cancer pathology in dogs using Raman spectroscopy of urine and chemometric analyses (ready for submission)). It has also shown success in detecting proteinuria in human urine and early stages of chronic kidney disease.

Embodiments of the invention include Aspect 1, which is a method of identifying and/or quantifying a condition of a subject comprising: obtaining a Raman spectrum from a sample from a subject; obtaining a transformed, baseline-corrected Raman spectrum by baselining and transforming the Raman spectrum by: (a) obtaining a baseline estimate on the Raman spectrum by fitting smoothing splines to the Raman spectrum; (b) determining a difference in intensity value at one or more wavenumber of the Raman spectrum as compared with a corresponding wavenumber of the baseline estimate; (c) obtaining an adjusted baseline estimate by adjusting the intensity values of the baseline estimate where a positive difference is determined, and where there is a zero or negative difference determined, then the intensity value of the initial baseline estimate remains the same; (d) iterating by repeating (a)-(c) on the adjusted baseline estimates; and (e) obtaining the transformed, baseline-corrected Raman spectrum by repeating (d) until a desired deviation between two consecutive adjusted baseline estimates is reached; optionally performing node optimization on the baseline-corrected Raman spectrum; analyzing the baseline-corrected Raman spectrum to detect the presence of and/or quantify a condition of the subject by analyzing peak position, height and/or area under the curve of one or more peaks of interest.

Aspect 2 is the method of Aspect 1, wherein the analyzing comprises one or more of principal component analysis (PCA), discriminant analysis of principal components (DAPC), partial least squares (PLS), and/or artificial neural networks (NN) to detect and/or quantify the condition.

Aspect 3 is the method of Aspect 1 or 2, wherein the analyzing comprises determining whether the baseline-corrected Raman spectrum is classified as being (a) from a subject who has the specified condition or (b) from a subject who does not have the specified condition and is performed in a manner such that it is determined that baseline-corrected Raman spectrum fits closer mathematically to one or the other statistically significant groups (a) or (b).

Aspect 4 is the method of any of Aspects 1-3, wherein the condition of the subject is any one or more of Bladder cancer (all types, grades, and stages); Acute cystitis (all types, grades, stages, and etiologies, including infectious and non-infectious etiologies); Chronic cystitis (all types, grades, stages, and etiologies, including infectious and non-infectious etiologies); Schistosomiasis; Kidney cancer (all types, grades and stages); Prostate cancer (all types, grades, and stages); Prostatitis (acute and chronic); Cervical cancer (all types, grades, and stages); Uterine cancer (all types, grades, and stages); Ovarian cancer (all types, grades, and stages); Cancer of the adrenal gland (all types, grades, and stages); Cushing's disease and Cushing's syndrome; Multiple myeloma with Bence-Jones proteinuria (all stages and grades); Acute kidney injury (all types and etiologies); Acute kidney failure (all types and etiologies); Chronic kidney failure (all types, stages, and etiologies); Acute glomerulonephritis (all types and etiologies); Chronic glomerulonephritis (all types and etiologies); Focal and diffuse segmental glomerulosclerosis (all stages, grades, and etiologies, including hypertension); Membranous nephropathy (all stages, grades, and etiologies); Membranoproliferative glomerulonephritis (all stages, grades, and etiologies, including systemic lupus erythematosus); Hemolytic uremic syndrome; IgA nephropathy (all stages, grades, and etiologies); Minimal change nephropathy (all stages, grades, and etiologies); Congenital nephropathy (all stages, grades, and etiologies); Diabetes; Diabetic nephropathy; Protein-losing nephropathy and nephrotic syndrome (all stages, grades, and etiologies); Acute pyelonephritis (all stages, grades, and etiologies); Chronic pyelonephritis (all stages, grade, and etiologies); Lyme disease (all stages and clinical presentations); Atypical borreliosis; Myalgic Encephalomyelitis/Chronic Fatigue Syndrome (ME/CFS) (all types, stages, and etiologies); Systemic mold allergy/toxicity; Hemobartonellosis; SARS-CoV-1 (Severe Acute Respiratory Syndrome Coronavirus Disease); SARS-CoV-2 (COVID-19 Disease); and MERS-CoV-2 (Middle Eastern Respiratory Syndrome Disease).

Aspect 5 is the method of any of Aspects 1-4, wherein the presence of the condition of the subject is made visible in the baseline-corrected Raman spectrum by emphasizing one or more of the peaks of interest and/or minimizing other peak(s).

Aspect 6 is a method of producing a transformed, baseline-corrected Raman spectrum comprising: applying an iterative fitting procedure to a Raman spectrum to adjust for peak invasion; in each iteration, prediction errors are adjusted down through a root transformation and added back to fitted baseline intensities to form a new set of intensities; applying smoothing splines to the new set of intensities to obtain a new baseline estimate, based on which a new set of prediction errors are calculated; and repeating the fitting procedure and stopping when errors fall below a desired level, such that a transformed, baseline-corrected Raman spectrum is obtained.

Aspect 7 is the method of Aspect 6, wherein the fitting procedure comprises adjusting intensity values of the fitted baseline intensities and/or the new set of intensities where a positive difference is determined, and where there is a zero or negative difference determined, then the intensity value remains the same.

Aspect 8 is a method of producing a transformed, baseline-corrected Raman spectrum comprising: (a) obtaining a baseline estimate on a Raman spectrum by fitting smoothing splines to the Raman spectrum; (b) determining a difference in intensity value at one or more wavenumber of the Raman spectrum as compared with a corresponding wavenumber of the baseline estimate; (c) obtaining an adjusted baseline estimate by adjusting the intensity values of the baseline estimate where a positive difference is determined, and where there is a zero or negative difference determined, then the intensity value of the initial baseline estimate remains the same; (d) iterating by repeating (a)-(c) on the adjusted baseline estimates; and (e) obtaining a transformed, baseline-corrected Raman spectrum by repeating (d) until a desired deviation between two consecutive adjusted baseline estimates is reached.

Aspect 9 is the method of Aspect 8, wherein the adjusting of the intensity values is performed according to:

$${y}_{i}^{( new )} = {\hat{m}}_{i} + \sqrt[4]{{δ}_{i}} ,$$

wherein: yᵢ^{(new)} is an adjusted intensity value; the difference in intensity value δᵢ is defined as: δᵢ=yᵢ−\hat{m}ᵢ; yᵢ is an intensity value of the Raman spectrum; and \hat{m}ᵢ is an intensity value of the baseline estimate.

Aspect 10 is the method of Aspect 8 or 9, wherein the iterating (d) is repeated until the desired deviation falls below a selected convergence criterion ε.

Aspect 11 is the method of Aspect 10, wherein the convergence criterion ε is a number in the range of from 0.0001 to 10.

Aspect 12 is the method of any of Aspects 8-11, wherein a number of knots (e.g. nodes) for the smoothing spline is a number in the range of from 5 to 20.

Aspect 13 is the method any of Aspects 8-12, wherein the Raman spectrum is from a fluid, tissue, gas, and/or solid.

Aspect 14 is the method of Aspect 13, wherein the Raman spectrum is from a dialysate or urine sample.

Aspect 15 is the method of any of Aspects 8-14, comprising: (a) obtaining an initial smooth baseline estimate by fitting smoothing splines to a raw Raman spectrum, where δᵢ=yᵢ−\hat{m}ᵢ is the deviation of the smooth baseline, \hat{m}ᵢ, from an observed raw spectral intensity, yᵢ; (b) adjusting intensities such that areas with zero or negative intensity deviations, δᵢ≤0, remain the same, while areas with positive intensity deviations, δᵢ>0, are updated as

$${y}_{i}^{( new )} = {\hat{m}}_{i} + \sqrt[4]{{δ}_{i}} ;$$

(c) feeding the new intensities, yᵢ^{(new)}, into the smoothing spline estimation again to get an updated baseline function estimate, \hat{ƒ}, and thus updated baseline intensity estimates \hat{m}ᵢ=\hat{ƒ}(i/n); (d) repeating one or more of steps (a)-(c) until a difference between two consecutive fitted baselines is small, and/or within a desired range, and/or below a desired limit, such as below a selected convergence criterion ε.

Aspect 16 is the method of any of Aspects 8-15, wherein the Raman spectrum is modeled as: y=m+a+ϵ, where m_{n×1}=(m₁, . . . , mₙ)^{T}, a_{n×1}=(a₁, . . . , aₙ)^{T}, and ϵ_{n×1}=(ϵ₁, . . . , ϵₙ)^{T} are respectively vectors of unknown true baseline intensities, peak intensities, and random noises.

Aspect 17 is a Raman spectrum baseline correction method, the method comprising: estimating a baseline of a Raman spectrum to obtain an estimated baseline, wherein the estimating is optionally performed by way of a smooth function; and removing the estimated baseline from the Raman spectrum, optionally by subtraction, to obtain a corrected Raman spectrum, wherein peak intensities in peak areas are preserved and intensities in non-peak areas are close to zero.

Aspect 18 is any of Aspects 1-17, wherein one or more peaks of interest are emphasized and/or one or more other peak(s) are minimized by way of node placement.

Aspect 19 is any of Aspects 1-18, wherein one or more of the knots/nodes are placed randomly and/or are placed to highlight regions of the Raman spectra in which one or more compound(s) of interest appears and/or are placed in a manner that highlights areas of interest in a urine spectrum to detect diabetes and/or are placed in a manner that highlight one or more areas of interest in a patient spectrum related to detecting the disease of hypertension.

Aspect 20 is any of Aspects 1-19, wherein node optimization is performed by placing a selected number of nodes to highlight regions of the Raman spectrum in which one or more compound(s) of interest appears and/or to highlight one or more regions where the Raman spectrum and the baseline do not overlap.

Aspect 21 is any of Aspects 1-20, wherein node optimization is performed such that a selected number of nodes are placed to highlight one or more areas of interest, and/or glucose, in a urine spectrum to detect diabetes.

Aspect 22 is any of Aspects 1-21, wherein node optimization is performed such that a selected number of nodes are placed to highlight one or more areas of interest in a patient spectrum related to and to detect hypertension.

Aspect 23 is any of Aspects 1-22, wherein knots for the smoothing spline are placed to highlight regions of the Raman spectrum in which one or more compound(s) of interest appears and/or are placed to highlight one or more regions where the Raman spectrum and the baseline do not overlap.

These and other aspects and embodiments of the invention are described below.

### BRIEF DESCRIPTION OF THE DRAWINGS

The accompanying drawings illustrate certain aspects of implementations of the present disclosure, and should not be construed as limiting. Together with the written description the drawings serve to explain certain principles of the disclosure.

FIGS. 1A-H are graphs showing baselining of a Raman spectrum: A-B) Goldindec; C-D) ISREA (with StaBAL—node optimization) node set 1; E-F) ISREA node set 2; and G-H) ISREA node set 3.

FIG. 2 is a graph showing hypothetical node placement in a Raman spectrum for the ISREA baseline application.

FIGS. 3A-B are graphs showing loss functions in baseline correction methods: A) Existing asymmetric loss functions (from top to bottom, the asymmetric Huber function as the dashed line, the asymmetric truncated quadratic function as the dash-dotted line, and the asymmetric Indec function as the dotted line) with the least squares loss function (solid line) imposed; and B) Proposed asymmetric root error loss function.

FIG. 4 is a diagram of an exemplary ISREA algorithm procedure.

FIGS. 5A-F are graphs comparing ALS, Goldindec, and ISREA (with NK=15) baseline correction on Raman spectra of six minerals against the expert-corrected spectra: A) Andersonite; B) Eastonite; C) Marialite; D) Parascholzite; E) Sugilite; and F) Wadeite.

FIGS. 6A-D are graphs comparing the ALS, Goldindec, and ISREA on dialysate spectra at 10 min of the session: A) All 50 dialysate spectra, NK=5; B) 10 dialysate spectra, NK=5; C) All 50 dialysate spectra, NK=10; and D) 10 dialysate spectra, NK=10. NK refers to number of knots selected.

FIGS. 7A-D are graphs comparing the ALS, Goldindec, and ISREA (with NK=5) on dialysate spectra at different time points in one treatment session. Graphs are shown at: A) 60 min; B) 120 min; C) 180 min; and D) 240 min of the session.

FIGS. 8A-L are graphs comparing Goldindec and ISREA where epsilon is: A-B) 10; C-D) 1; E-F) 0.1; G-H) 0.01; I-J) 0.001; and K-L) 0.0001.

### DETAILED DESCRIPTION OF VARIOUS EMBODIMENTS OF THE INVENTION

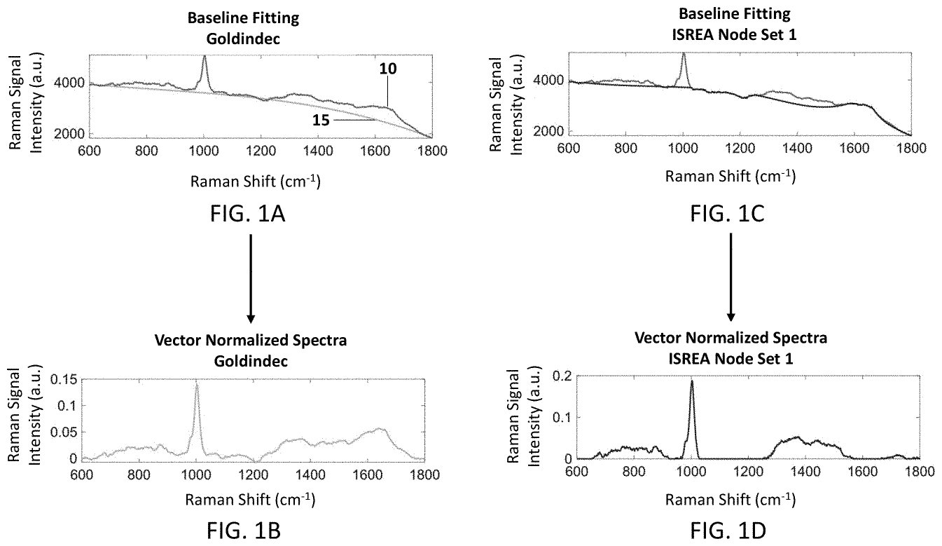

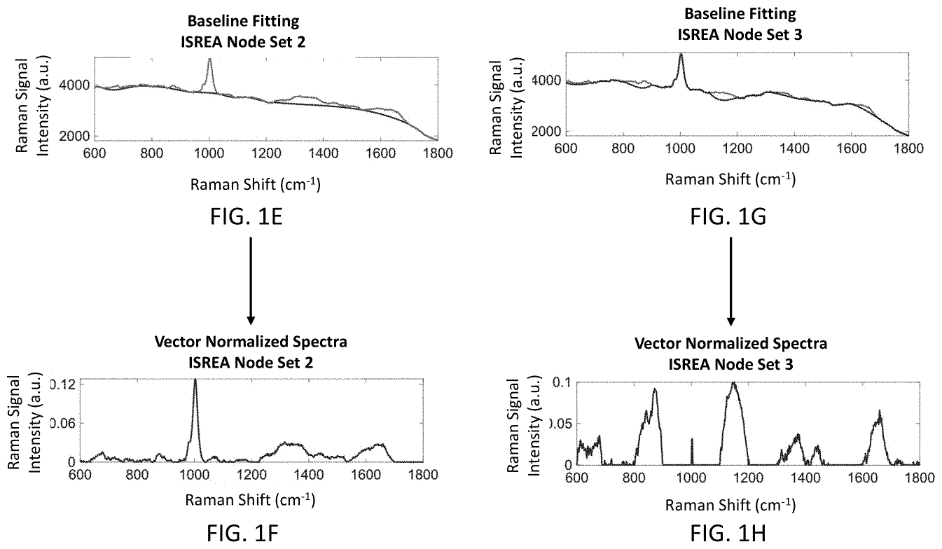

The present invention comprises a new computationally efficient algorithm, called the Iterative Smoothing-spline with Root Error Adjustment (ISREA). A typical Raman spectrum 10 is shown in FIG. 1A. Raman spectra are typically baselined to remove background fluorescence from the Raman signal. One method for doing this is the Goldindec algorithm. The Goldindec baseline 15 is shown in FIG. 1A. When this background is subtracted, the resulting spectrum is used for analysis (FIG. 1B). Several other methods, such as high-order polynomials, have been used for this purpose as well. The present invention, ISREA, also performs this function, but with much improved results.

In ISREA, the baseline is fitted by smoothing splines, given their better flexibility in capturing the overall shape of the spectrum. To correct the aforementioned peak-invading problem of the smoothing spline baseline estimate, the present inventors observed that peak invasion mostly happens in the regions of positive deviations or positive prediction errors, that is, the regions where the observed intensity deviates from the fitted baseline intensity by a positive amount. Therefore, the following iterative fitting procedures are employed to adjust for the peak invasion. In each iteration, the prediction errors are adjusted down through a root transformation and added back to the fitted baseline intensities to form a new set of intensities. Then smoothing splines are applied to this new set of intensities to obtain a new baseline estimate, based on which a new set of prediction errors are calculated. This adjustment procedure is repeated until the errors drop to a negligible level. The error transformation used in the adjustment is motivated from asymmetric loss functions where large positive deviations are penalized less than in a least square loss. So, the ISREA baseline estimate inherits favorable properties of those estimates obtained from asymmetric losses. Furthermore, the simple root error adjustment avoids the tricky optimization of a non-conventional objective function otherwise required in all the methods based on asymmetric losses. Therefore, it is much easier to implement, much more computationally efficient, and lends well to automated analyses.

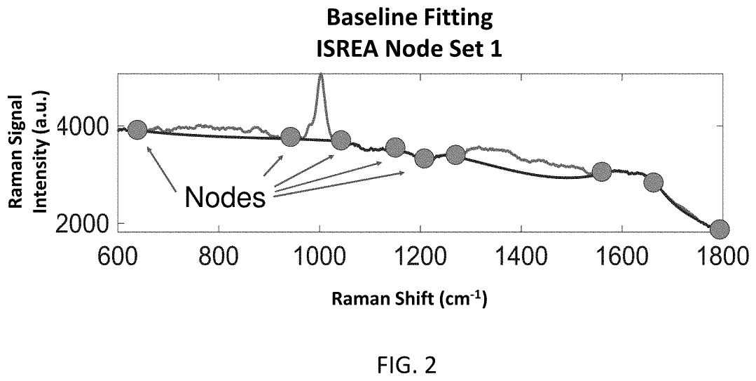

Rametrix™ originally used the Goldindec algorithm for removing background fluorescence and effectively “baselining” the spectra for direct comparisons. The Rametrix™ Signal Processing toolbox has since been updated to remove the Goldindec algorithm and replace it with ISREA. ISREA fits several cubic splines through a Raman spectrum (FIGS. 1C-H). The cubic splines attach to nodes (e.g. knots) placed in the Raman spectrum. This is illustrated for a hypothetical case in FIG. 2. In the original version of ISREA (Xu, Y, et. al. (2020): Xu, Y, Du, P, Senger, R, Robertson, J, Pirkle, J “ISREA: An efficient peak-preserving baseline correction algorithm for Raman spectra,” First Published Oct. 8, 2020 https://doi.org/10.1177/0003702820955245), these nodes were static in number and placement in a spectrum. The present invention advances this concept by instructing ISREA how many nodes to place in a spectrum and where to place them (shown in FIG. 2). The result of such ISREA node optimization is a baselined and transformed spectrum. In this example, the nodes were placed to highlight the regions where the spectrum and the baseline do not overlap. Where the lines overlap will show up as zero signal when the spectrum is transformed (spectrum minus baseline and normalized to total area=1). This enhances the regions where the spectrum and baseline do not overlap. Further, the implementation in FIG. 2 will highlight the regions between the left-most node (node 1) and node 2 (approx. 600-950 cm-1). It will highlight the region between nodes 2 and 3 (approx. 990-1040 cm-1) and between nodes 6 and 7 (approx. 1250 to 1550 cm-1).

ISREA with node optimization allows certain regions of a Raman spectrum to be emphasized and other areas to be minimized or disregarded altogether. Nodes are placed at various points within the spectrum to maximize differences between groups of spectra. Alternatively or in addition, node placement can be used to highlight areas that correspond to regions that are known to contain signals corresponding to chemicals/peaks of interest. After node placement, baselined and transformed spectra are analyzed by the Rametrix™ Data Analysis Toolbox, which includes principal component analysis (PCA), discriminant analysis of principal components (DAPC), partial least squares (PLS), and artificial neural networks (NN) to detect the presence of disease. ISREA with node optimization also enables a particular disease to become visible in transformed spectra. This is unique and a new advancement in Raman spectroscopy data analysis. FIGS. 1C-H provide further examples of spectrum transformation with ISREA given different node placements. FIG. 1C shows ISREA fitting of the same Raman spectrum using an optimized set of nodes to detect hematuria (i.e., blood) in human urine. In this example, the nodes were placed using blood-spiked samples of urine. The nodes were placed to highlight the regions of spectra that had signal intensities aligning with blood concentration. The resulting baselined spectrum is shown in FIG. 1D. This transformed spectrum eliminates superfluous Raman data present in FIG. 1B and has shown to greatly improve the detection of hematuria. Other node sets used with ISREA, are shown in FIGS. 1E-H. These demonstrate how different node placements can transform a Raman spectrum differently.

The present inventors have found that specific node sets exist for each disease detected. Thus, the combination of ISREA baselining with node optimization for spectrum transformation are unique aspects of Rametrix™ that enable the detection of disease in human urine. The technique is also applicable to other fluids (such as dialysate, blood, serum, plasma, saliva), tissues, gasses, and solids. It can enable identifying environmental toxins, materials properties, physiology changes, other pathologies, specific chemicals, such as explosives, and biomolecular/chemical changes in general.

In the following numerical experiments, ISREA is compared with the ALS (P. H. C. Eilers et al., 2003) and Goldindec algorithm (J. Liu et al., 2015) on both simulated and real Raman spectral data. In simulations, both spiky (e.g. samples with sharp and prominent peaks) and non-spiky (e.g. samples with low peaks) data were considered. In real applications, spectra of pure mineral data from public databases and spectra of waste dialysate samples collected from patients undergoing hemodialysis treatments in a clinic were studied. The spectrum of a pure mineral generally contains a small number of Raman peaks. Additionally, there is an expert-corrected spectrum available so that a “true” baseline can be recovered as the reference. On the other hand, a waste dialysate sample is a complicated solution containing many molecules, so its spectrum is expected to contain many Raman peaks. Furthermore, there is no ground truth or expert correction available as the reference, although some signature chemicals such as urea are always present in the sample. The experiments described herein demonstrate that ISREA is adaptive to spectra with sparse or dense Raman peaks and has better performance on both simulated and real data.

Method

Notation and Asymmetric Losses for Baseline Correction

A raw Raman spectrum consists of a sequence of intensity measurements y at Raman shifts or wavenumbers (in cm⁻¹), i=1, . . . , n. Let y_{n×1}=(y₁, . . . , yₙ)^{T} be the vector of observed intensities. The model for a raw Raman spectrum is

y=m+a+ε (1)

where m_{n×1}=(m₁, . . . , mₙ)^{T}, a_{n×1}=(a₁, . . . , aₙ)^{T} and ε_{n×1}=(ε₁, . . . , εₙ)^{T}, are respectively vectors of unknown true baseline intensities, peak intensities, and random noises. In particular, the baseline intensities mᵢ are often assumed to come from an unknown smooth baseline function ƒ, say, defined on the interval [0, 1], such that mᵢ=ƒ(i/n). The goal of a baseline correction procedure is thus to recover this unknown baseline function ƒ.

While smoothers, such as polynomials or splines, are often used to model the baseline function ƒ, a proper model for peak intensities aᵢ is extremely hard to achieve. Each peak in the spectrum represents a specific molecular structure present in the scanned sample. When all the chemical compositions of the sample are known, the peaks can be modeled as properly-sized spikes at expected wavenumbers on the spectrum. In practice, the composition of the sample (e.g., the waste dialysate sample in the hemodialysis experiment) is often unknown, making it difficult or even impossible to model the peak intensities properly. Instead, most baseline correction methods simply build up a smoother through the minimization of a loss function without any explicit modeling of peak intensities aᵢ. That is, the baseline is estimated essentially with peak intensities a, absorbed into the random error part of model in Eq. (1). For example, when the least squares loss Lₗₛₑ, (x)=x² is used, a smoother is trained through the minimization of Σₙ₌₁ⁿ=δᵢ² where δᵢ=yᵢ−\hat{m}ᵢ is the deviation of the fitted baseline from the observed intensity, and \hat{m}ᵢ=\hat{ƒ}(i/n) is the fitted baseline intensity derived from the smooth function estimate \hat{ƒ} of ƒ.

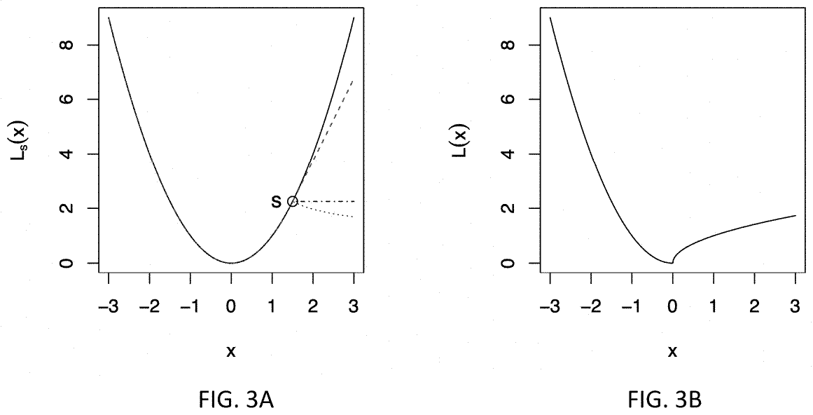

As previously discussed, the baseline function estimate \hat{ƒ} based on the least squares loss often cuts into the peak areas. To address this issue, various asymmetric loss functions Lₛ(x), where s>0 is a pre-specified threshold, are introduced such that Lₛ (x)=x² when x<s but Lₛ (x)<x² for x≥s. The principle for such a setup is to reduce the punishment otherwise enforced by the least squares loss when the difference δᵢ is a big positive number, a phenomenon often observed at the peak area. For example, FIG. 3A shows several such asymmetric loss functions. From top to bottom the curves are respectively the asymmetric Huber function with Lₛ(x)=2sx−x² when x≥s, the asymmetric truncated quadratic function with Lₛ(x)=s² when x≥s, and the asymmetric Indec function with

$${L}_{s} ( x ) = \frac{{s}^{3}}{2 x} + \frac{{s}^{2}}{2}$$

when x≥s.

These asymmetric loss functions all share the same shape with the least squared loss for negative deviations while they have reduced loss for large positive deviations. This helps discourage invasions into the high peaks commonly seen in a baseline minimizing the least squares loss. However, these asymmetric loss functions have a couple of critical drawbacks: (1) the selection of threshold s can be tricky; (2) the optimization of these loss functions is often time-consuming due to their nonstandard forms. To address the issue of threshold selection, we propose the following threshold-free asymmetric root error loss function:

$$L ( x ) = \{ \begin{matrix}{x}^{2} , if x \leq 0 \\ \sqrt{x} , if x > 0\end{matrix} \qquad ( 2 )$$

As plotted in FIG. 3B, the asymmetric square root error function clearly preserves the discouragement of large positive deviations. And it has the advantage of not requiring any manual selection of a threshold. To further improve the computational efficiency, the direct optimization of the loss function in Eq. (2) is avoided. Instead, a computational procedure that iterates between smoothing and updating “observations” with positive deviations by the sums of current smooth baseline estimates and square-root-adjusted deviations is employed.

The ISREA Algorithm

The present inventors now introduce the Iterative Smoothing-splines with Root Error Adjustment (ISREA) algorithm. The basic idea is to yield a baseline function estimate that targets at minimizing a loss function similar to Eq. (2) without really resorting to its direct optimization which can be complicated and time consuming.

In the ISREA algorithm, an initial baseline estimate is first obtained by fitting smoothing splines to the raw spectrum. Recall that δᵢ=yᵢ−\hat{m}ᵢ is the deviation of the smooth baseline estimate \hat{m}ᵢ from the observed raw spectral intensity value yᵢ. Then, intensities are adjusted in a way such that in the areas with δᵢ<0 the intensities remain the same, while in the areas with δᵢ>0 intensities are updated as

$${y}_{i}^{( new )} = {\hat{m}}_{i} + \sqrt[4]{{δ}_{i}} .$$

To see how this adjustment is related to the asymmetric root error loss function L(x) in Eq. (2), we note that squared errors at areas with non-positive deviations remain δⱼ² while squared errors at areas with positive deviations become \sqrt{δᵢ}. The new intensities yᵢ^{(new)} are then fed into smoothing spline estimation again to get an updated baseline function estimate \hat{ƒ} and thus updated baseline intensity estimates \hat{m}ᵢ=\hat{ƒ}(i/n).

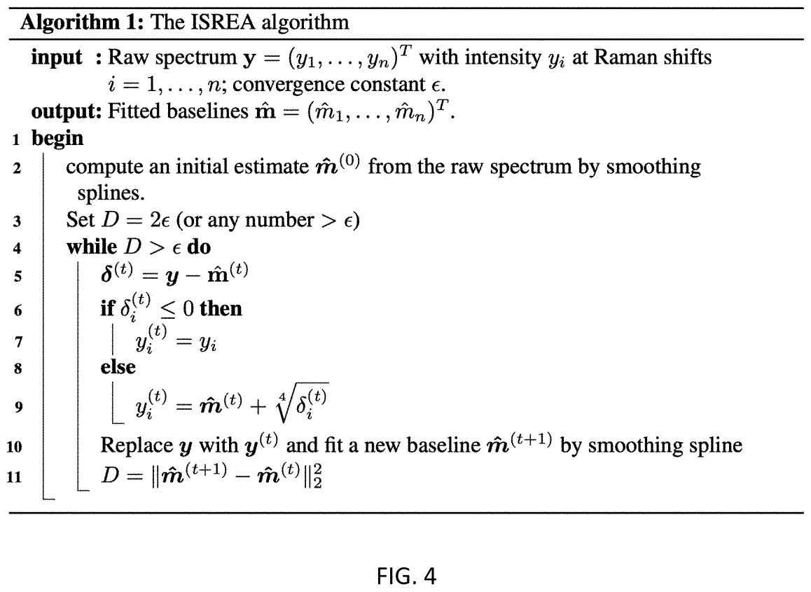

These steps are repeated until the difference between two consecutive fitted baselines is small. The complete algorithm of ISREA is shown in FIG. 4.

In each iteration, intensities at peak areas are reduced and intensities at non-peak areas remain the same. So smoothing splines are actually fitted to a modified spectrum with reduced peak heights. Consequently, the fitted baseline can stay low at the bottom of peaks like the true baseline, and provides a properly corrected spectrum with retained peak heights.

Function Smooth.spline in the R package stats is used to fit a cubic smoothing spline to the data. For the number of knots, 5 are used for general raw spectra and 15 for really spiky raw spectra. However, the number of knots (e.g. nodes) can be any integer from 5 to 20, including 5, 6, 7, 8, 9, 10, 11, 12, 13, 14, 15, 16, 17, 18, 19, or 20 nodes/knots or more, such as from above 0 to 5 nodes/knots, or from 2-15 node/knots, or from 10-25 nodes/knots, or from 15-20 nodes/knots. In some embodiments, the number of knots can exceed 20, such as 25, 30, 50, or more knots. For the constant ε in the convergence criterion, different choices of ε have been tested in a range from 10 to 0.0001 on simulated spectra, dialysis spectra, and mineral spectra. The results, collected in the supplemental material, are quite similar and do not appear to be sensitive to its choice. More time is taken as ε decreases since more iterations are needed.

Numerical Studies

In this section, the ISREA is compared with two existing baseline correction methods, the ALS and Goldindec. For the ALS, the “als” method of the baseline function in the R package baseline with the options lambda=7.5 and weight=0.05 is used, as found by optimizing baseline accuracy over a grid of plausible values (H. F. M. Boelens, P. H. C. Eilers. Baseline correction with asymmetric least squares smoothing. Leiden University Medical Centre report. 2005). For the Goldindec, degree three polynomials and a peak ratio of 0.5 are used, as suggested in their paper (J. Liu et al., 2015). The ISREA is tested with different choices of the number of knots.

### Example 1: Simulated Spectra

Simulated data were generated from the assumed Raman spectrum model Eq. (1). In the simulation, the true baseline intensity m was set to be a polynomial function of degree five whose coefficients were generated from Normal distributions. The first five coefficients were generated from N(0, 10²) while the last one was generated from N(0, 0.01²). Peaks were simulated from groups of Gaussian distributions, with the number of them randomly selected between one to ten. Their central locations were randomly set within the spectral range. Standard deviations of those peaks were independently selected in the range from one to fifteen such that the width of those artificial peaks was similar to peaks on real Raman spectra. Noise signals were generated from N(0,1). One thousand Raman spectra were simulated this way and tested with the ISREA and Goldindec.

A statistical measure, called AC_rate, was used to evaluate the performance of ISREA. It was proposed by Liu et al (J. Liu et al., 2015). to assess the accuracy of the Goldindec method and defined as:

$$AC\_rate = 1 - \frac{{‖ m - \hat{m} ‖}_{2}}{{‖ m ‖}_{2}} \qquad ( 3 )$$

where m represents the true/expert-fitted baseline intensity, \hat{m} represents the fitted baseline intensity, and ∥·∥₂ is the L² norm of a function on the spectral range. AC_rate compares the true/expert-corrected baseline with the fitted baseline. Generally, a bigger AC rate that is close to 1 is desired. Besides AC_rate, the root mean square error (RMSE) between the true and estimated baselines was considered (S. He et al., 2014). For simulated data and minerals data, AC_rate and RMSE were used to compare the ALS, ISREA and Goldindec baseline estimates against the true baselines or expert-corrected baselines. For dialysis data, since there are no true/expert-correction baselines, baseline-corrected spectra of dialysis samples to compare the ALS, ISREA and Goldindec methods were plotted.

Table 1 and Table 2 present the AC_rate and RMSE percentiles for the ALS, Goldindec and ISREA with different numbers of knots (NK). While the Goldindec and ALS methods yielded a satisfactory AC_rate for 50% of the simulated spectra, it should also be noted that the AC_rate for the lower 25% of the spectra is much deteriorated. In particular, the smallest AC_rates were even negative for both methods, indicating a complete miss of the true baseline. On the other hand, the AC_rates of the ISREA with different numbers of knots are consistently better than those of the Goldindec and ALS. In terms of the RMSEs in Table 2, the ISREA completely dominated the other two methods with much smaller RMSEs. In general, although NK=5 generally gave the best performance for ISREA, the AC_rates and RMSEs for NK=10 and NK=15 were very close. This indicates that the ISREA method is not sensitive to different choices of the number of knots.

TABLE 1
Percentiles of the AC_rates for ALS, Goldindec and ISREA on
1000 simulated spectra. NK is the number of knots used in ISREA.

| Percentile | | 0% | 5% | 25% | 50% | 75% | 95% | 100% |
|---|---|---|---|---|---|---|---|---|
| ALS | | −0.6198 | 0.7748 | 0.9322 | 0.9555 | 0.9789 | 0.9853 | 0.9896 |
| Goldindec | | −0.8795 | 0.6959 | 0.9107 | 0.9515 | 0.9700 | 0.9833 | 0.9895 |
| ISREA | NK = 5 | 0.6909 | 0.9355 | 0.9843 | 0.9935 | 0.9965 | 0.9985 | 0.9995 |
| | NK = 10 | 0.6178 | 0.9214 | 0.9812 | 0.9921 | 0.9957 | 0.9981 | 0.9994 |
| | NK = 15 | 0.5832 | 0.9123 | 0.9785 | 0.9906 | 0.9950 | 0.9979 | 0.9992 |

TABLE 2
Percentiles of the RMSE for ALS, Goldindec, ISREA on 1000 simulated
spectra. NK is the number of knots used in ISREA.

| Percentile | | 0% | 5% | 25% | 50% | 75% | 95% | 100% |
|---|---|---|---|---|---|---|---|---|
| ALS | | 0.85 | 0.95 | 1.10 | 1.31 | 1.80 | 2.75 | 4.80 |
| Goldindec | | 0.90 | 1.13 | 1.27 | 1.55 | 2.36 | 4.10 | 15.32 |
| ISREA | NK = 5 | 0.05 | 0.10 | 0.18 | 0.27 | 0.35 | 0.45 | 0.68 |
| | NK = 10 | 0.08 | 0.13 | 0.22 | 0.33 | 0.42 | 0.54 | 0.76 |
| | NK = 15 | 0.08 | 0.15 | 0.26 | 0.38 | 0.48 | 0.61 | 0.91 |

### Example 2: Mineral Spectra

In this section, the ALS, Goldindec and ISREA methods are compared on Raman spectra of minerals obtained from the RRUFF database (B. Lafuente, R. T. Downs, Y. H, S. N. “The power of databases: the RRUFF project”. In: T. Armbruster, R. M. Danisi, editors. Highlights in Mineralogical Crystallography. W. De Gruyter, Berlin, Germany, 2015. Pp. 1-30, incorporated by reference herein in its entirety). The database provides both the raw spectra and expert-corrected spectra for many minerals, the differences of which can be treated as the “true” baselines.

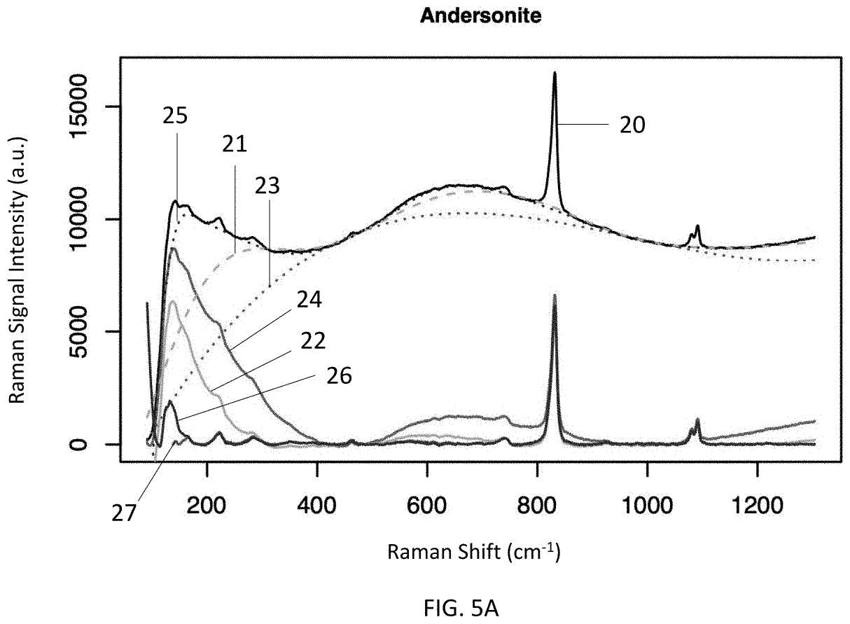

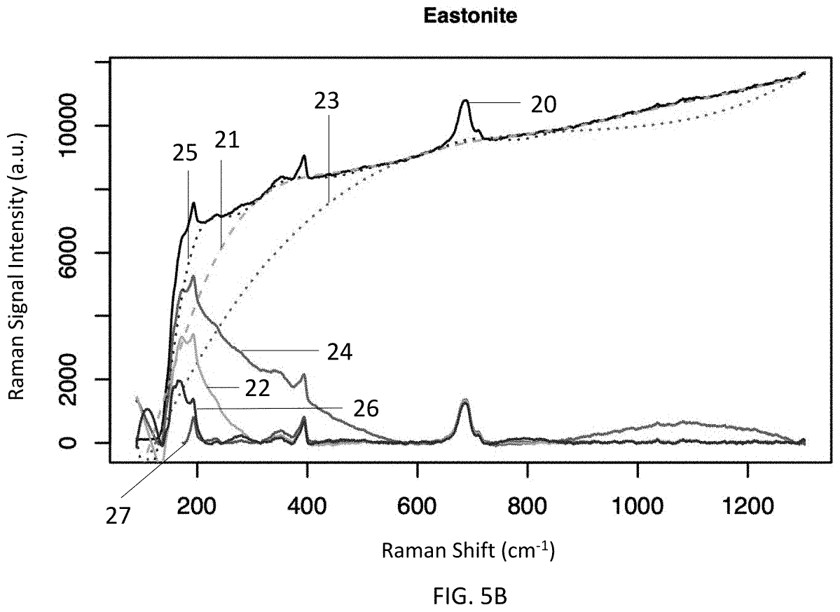

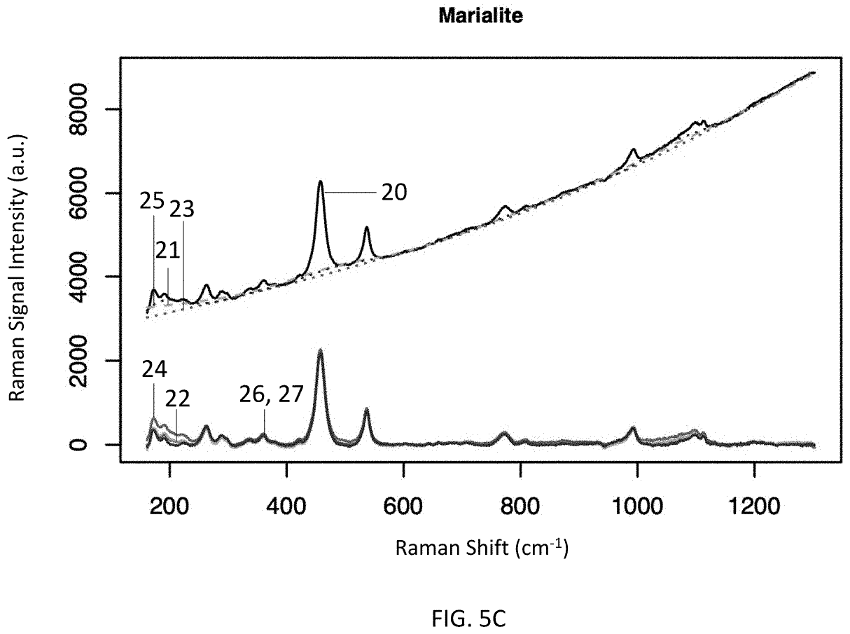

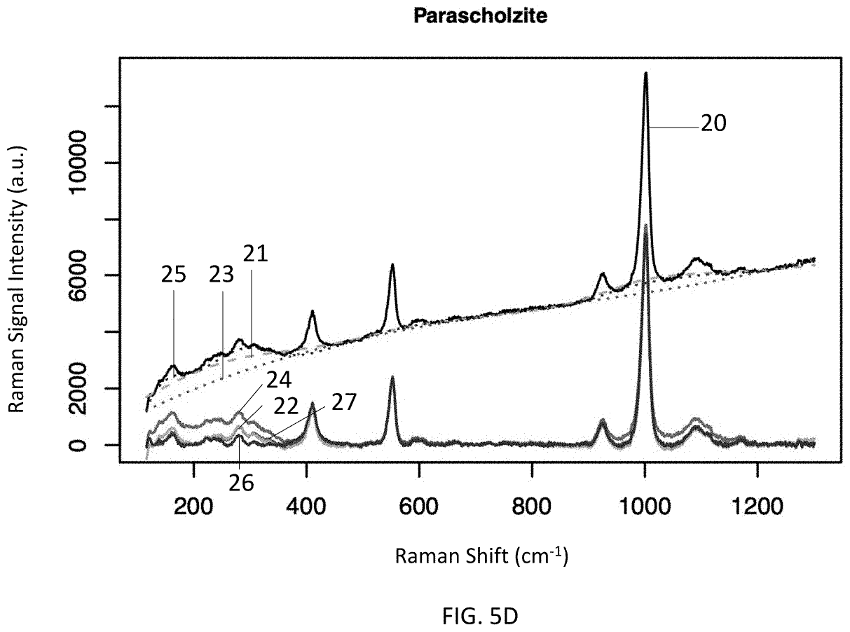

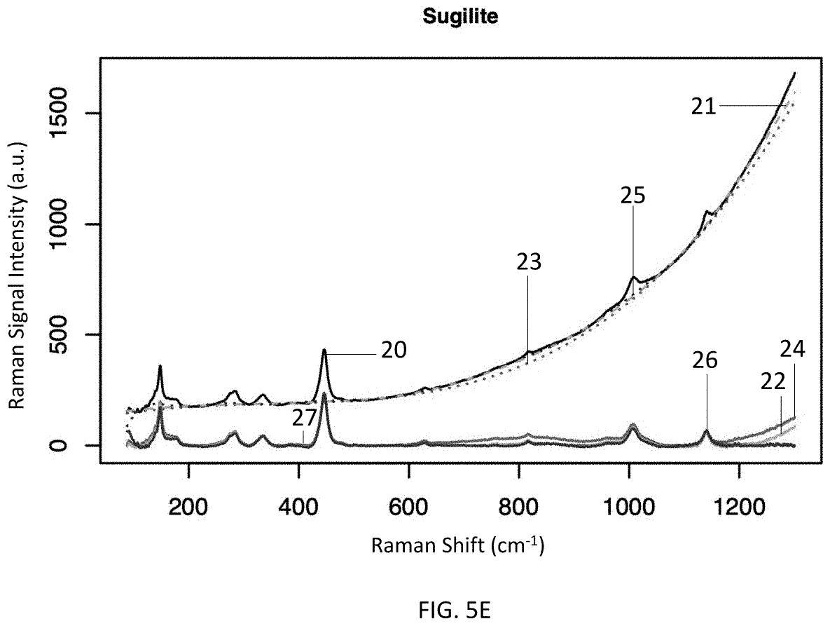

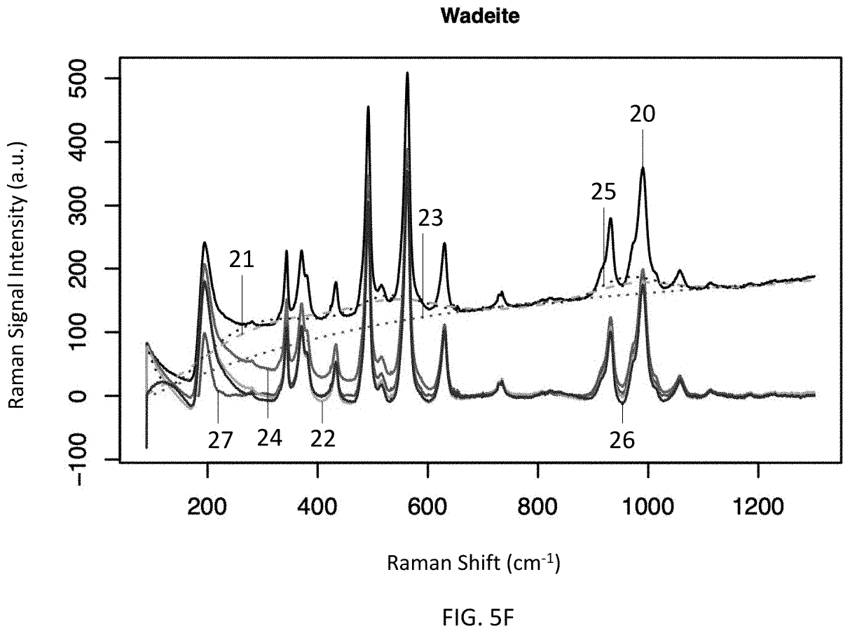

The ALS, ISREA and Goldindec methods were applied to spectra of six minerals, namely, andersonite, eastonite, marialite, parascholzite, sugilite, and wadeite. These six minerals, two of which also appeared in the Goldindec paper (J. Liu et al., 2003), were selected to represent some typical variations of Raman peak locations (in the middle vs. at the ends of the spectral domain), sharpness (sharp peaks vs. low peaks), and spread (clustered peaks vs. spread-out peaks). Compositions of these minerals are simple and pure. Therefore, their Raman spectra are also simple with clear Raman peaks. FIGS. 5A-F compare the three versions of baseline corrected spectra against the expert-corrected spectra 27. The curves are respectively: raw spectra 20, ALS baseline estimates 21, ALS-corrected spectra 22, Goldindec baseline estimates 23, Goldindec-corrected spectra 24, ISREA baseline estimates 25, and ISREA-corrected spectra 26. Clearly, both the ALS and the ISREA-corrected spectra almost overlap the expert-corrected spectra for all the six minerals, whereas Goldindec sometimes generated spectra that deviated from the expert-corrected spectra by large margins at parts of the spectral domain. Further examination of the AC_rates in Table 3 and RMSEs in Table 4 confirmed that Goldindec did not perform as well in baseline correction for three of the six minerals, namely, andersonite, eastonite, and wadeite. A close inspection of the raw spectra for these minerals revealed that their underline baselines have complicated shapes which are generally difficult to capture with polynomials. Next, the ALS is compared with the ISREA. In terms of the AC_rates in Table 3, the ALS and ISREA had similar AC_rates although the ISREA with different choices of NK had slightly higher AC_rates for five of the six minerals, except for parascholzite where both methods essentially had the same AC_rate. In terms of the RMSEs in Table 4, the ISREA with NK=15 had smaller RMSEs than the ALS for five of the six minerals, except for parascholzite where the ALS had a slightly lower RMSE.

TABLE 3
AC_rate comparisons of ALS, Goldindec and ISREA for six minerals.

| | | Andersonite | Eastonite | Marialite | Parascholzite | Sugilite | Wadeite |
|---|---|---|---|---|---|---|---|
| ALS | | 0.8891 | 0.9412 | 0.9935 | 0.9825 | 0.9815 | 0.8916 |
| Goldindec | | 0.7822 | 0.8546 | 0.9878 | 0.9413 | 0.9598 | 0.8044 |
| ISREA | NK = 5 | 0.8789 | 0.9479 | 0.9936 | 0.9740 | 0.9923 | 0.8910 |
| | NK = 10 | 0.9653 | 0.9484 | 0.9966 | 0.9806 | 0.9913 | 0.9055 |
| | NK = 15 | 0.9872 | 0.9818 | 0.9951 | 0.9824 | 0.9875 | 0.8938 |
| | NK = 20 | 0.9800 | 0.9884 | 0.9924 | 0.9814 | 0.9823 | 0.9152 |
| | NK = 25 | 0.9861 | 0.9805 | 0.9888 | 0.9717 | 0.9781 | 0.9140 |

TABLE 4
RMSE comparisons of ALS, Goldindec and ISREA for six minerals.

| | | Andersonite | Eastonite | Marialite | Parascholzite | Sugilite | Wadeite |
|---|---|---|---|---|---|---|---|
| ALS | | 1075.25 | 559.36 | 36.96 | 81.85 | 11.41 | 16.02 |
| Goldindec | | 2110.84 | 1382.52 | 69.68 | 274.02 | 24.74 | 28.90 |
| ISREA | NK = 5 | 1174.22 | 494.93 | 36.68 | 121.57 | 4.77 | 16.11 |
| | NK = 10 | 336.14 | 491.04 | 19.35 | 90.67 | 5.37 | 13.96 |
| | NK = 15 | 124.54 | 173.34 | 27.91 | 82.24 | 7.72 | 15.69 |
| | NK = 20 | 194.33 | 110.57 | 43.78 | 86.93 | 10.92 | 12.53 |
| | NK = 25 | 135.10 | 185.63 | 63.91 | 131.90 | 13.47 | 12.71 |

In numerical experiments, different choices of NK for the ISREA were also considered. As Tables 3 and 4 show, in general ISREA is not sensitive to the choice of NK with different NKs yielding similar AC_rates and RMSEs. This confirmed the findings of the previous simulations. One noticeable exception is the NK=5 case for andersonite (FIG. 5A), where the sharp peak at the left end of the raw spectrum might be hard to capture when the number of knots is too small. As demonstrated by the results in Tables 3 and 4, this can be easily handled by using a slightly larger number of knots.

### Example 3: Dialysate Spectra

The last set of spectra were collected in the hemodialysis experiment. Hemodialysis is one of the most common treatments for patients with end stage kidney diseases. In a hemodialysis treatment session, typically about 4 hours long, a machine called dialyzer is connected to the patient to help partially purify blood by removing metabolic waste products and rebalancing water and electrolytes. The dialyzer pumps the blood to a filter chamber, composed of a semipermeable polysulfone membrane that separates the patient's blood from a fluid called dialysate. Wastes in the blood are released to the dialysate in the chamber, as fresh blood flows out of the chamber.

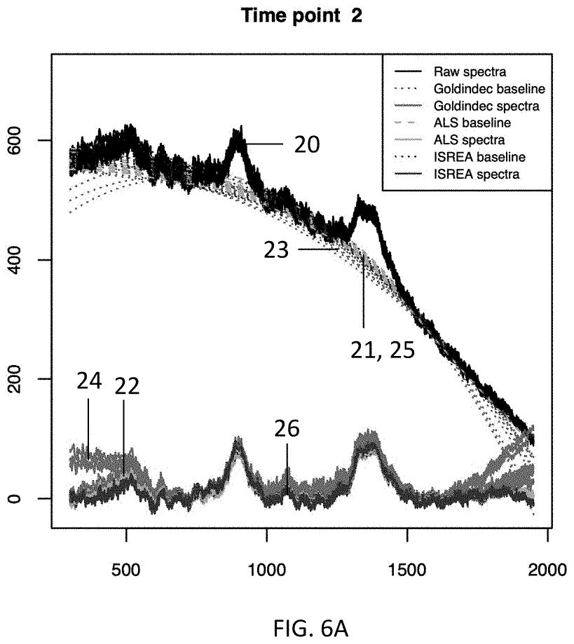

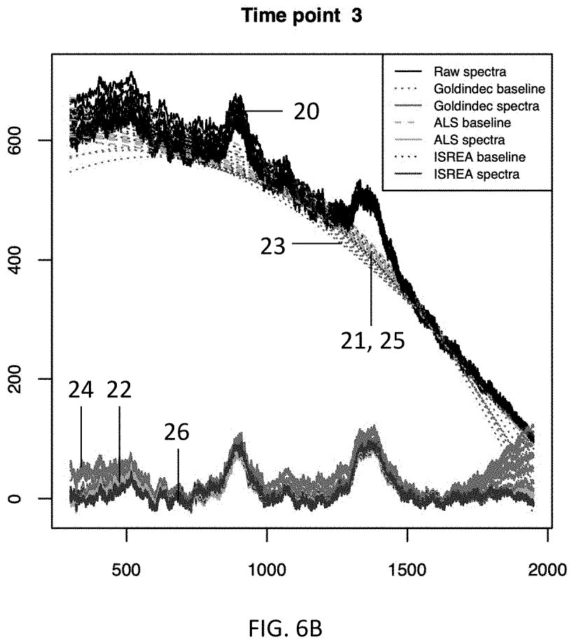

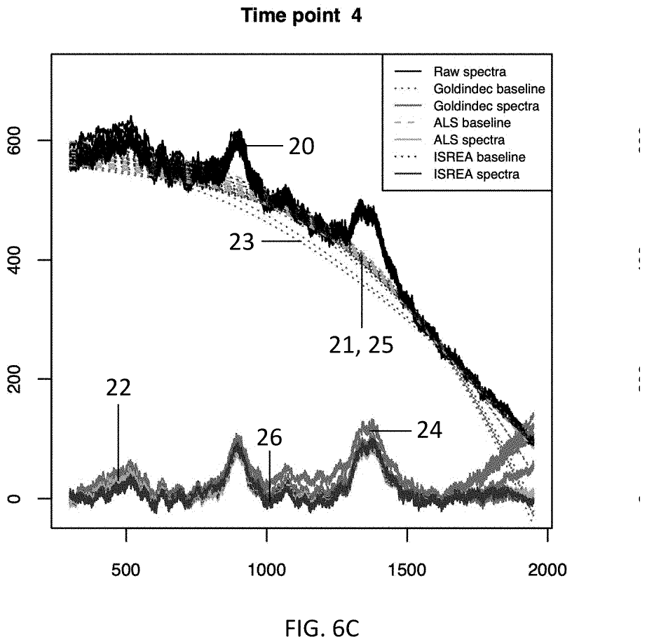

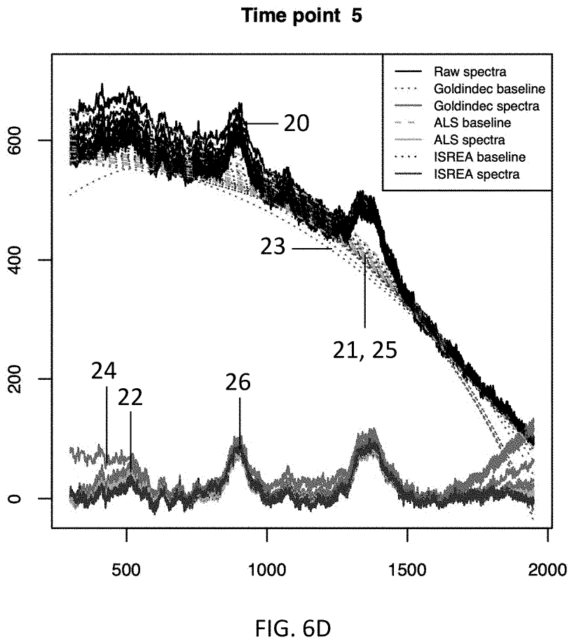

The hemodialysis study consisted of a total of 30 treatment sessions for 10 chronic kidney disease patients. The analysis of the Raman spectral data from one treatment session is now described. The analysis of other treatment sessions yielded similar results. Used dialysate samples were collected (containing metabolic wastes) at 10 min, 60 min, 120 min, 180 min, and 240 min (the end) of the session (FIGS. 6A-D). Each sample was divided into 10 portions and each portion was analyzed by a Raman spectrometer (Peakseeker Pro 785, Agiltron Inc., Woburn, Mass.) to produce a raw spectrum. Therefore, a total of 50 raw Raman spectra were generated with 10 spectra associated with each time point. As discussed earlier, there were no expert-corrected spectra of waste dialysate for references.

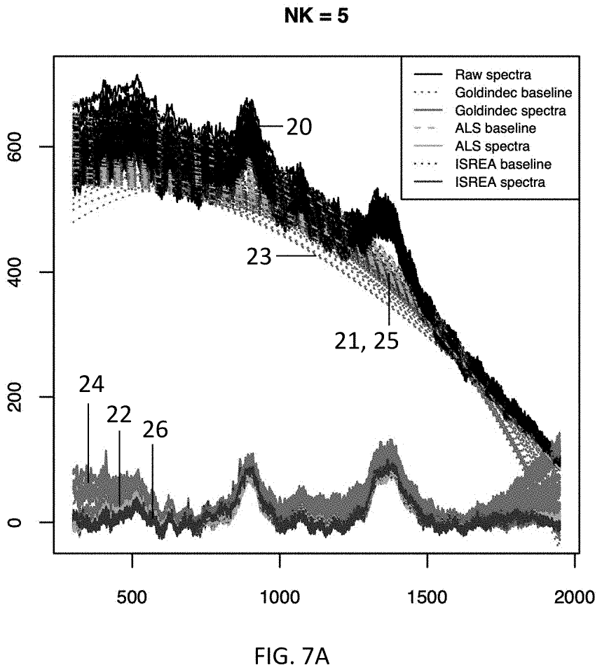

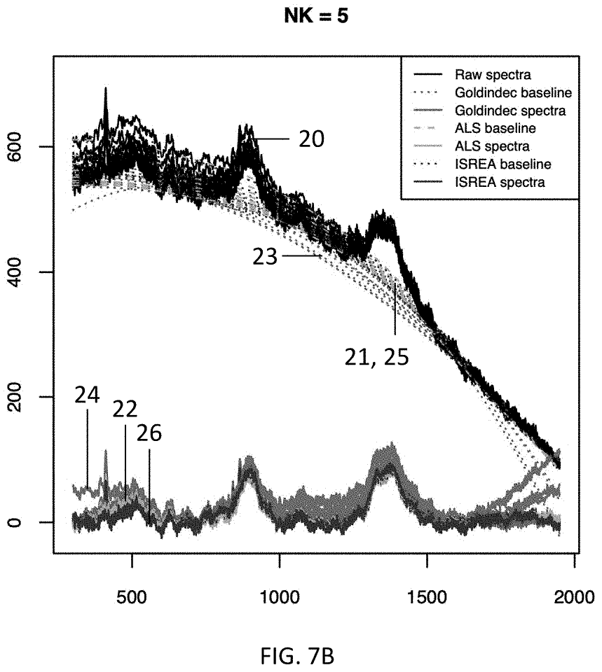

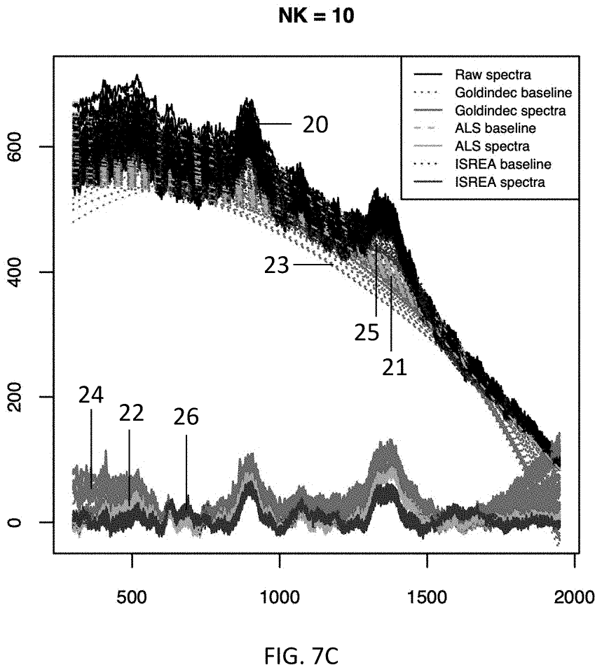

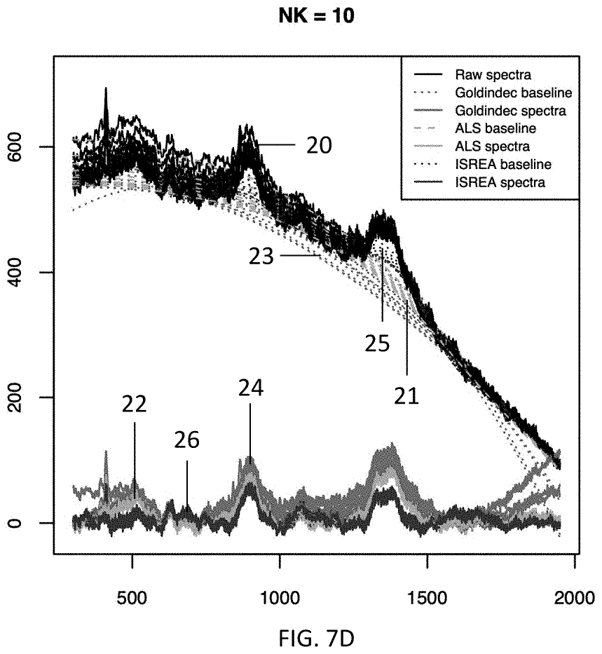

Visual comparisons of ALS, Goldindec and ISREA are displayed in FIGS. 7A-D. Clearly, both NK=5 (FIGS. 7A-B) and NK=10 (FIGS. 7C-D) for ISREA produced consistent baseline estimates and yielded similar baseline-corrected spectra for the 50 raw spectra. ALS also generated consistent baseline estimates, with slightly lower peaks than those from ISREA. On the other hand, a good portion of the Goldindec baselines completely missed the trend at the right end of the spectral domain. Since peaks in this area can be associated with important molecules in used dialysate, this can cause serious trouble for ensuing quantitative analysis of the compositions of used dialysate samples.

To study this further, how much ALS, ISREA and Goldindec respectively preserved the similarities between spectra was also numerically examined. As shown in FIGS. 7A-D, since all the raw spectra were from used dialysate samples collected in the same treatment session, they all displayed similar trends. Such similarity is expected to be preserved even after the baseline correction. To represent this similarity, the first raw spectrum was paired with each of the other raw spectra, resulting in 49 pairs of spectra. For each pair, the correlation between the intensities of the two spectra was calculated. Then these 49 correlations represented the similarity between the 50 raw spectra. Similarly, 49 correlations each were obtained for the ALS baseline-corrected spectra, the Goldindec baseline-corrected spectra, the ISREA baseline-corrected spectra with NK=5, and the ISREA baseline-corrected spectra with NK=10. The similarity changes were then calculated by subtracting each of these 3 sets of correlations for baseline corrected spectra from the correlations for raw spectra. Hence, these correlation differences represented the similarity changes after each baseline correction procedure. The means and standard deviations of these correlation differences are summarized in Table 5. It shows that Goldindec baseline correction dramatically decreased the similarities between raw spectra, whereas ALS and the ISREA baseline correction procedure with NK=5 or NK=10 preserved the similarities between raw spectra much better. The mean similarity difference was the smallest for the ISREA with NK=5 while the mean similarity difference for the ALS was only slightly larger.

TABLE 5
Similarity change
comparisons of
ALS, Goldindec and ISREA on
dialysate spectra.

| | | | Standard |
|---|---|---|---|
| | | Mean | deviation |
| ALS | | 0.0507 | 0.0082 |
| Goldindec | | 0.2559 | 0.2282 |
| ISREA | NK = 5 | 0.0446 | 0.0072 |
| | NK = 10 | 0.1238 | 0.0206 |

Computational Cost Comparison

Another advantage of ISREA is its relatively low computational cost, making it ideal for efficient batch processing of a large number of raw Raman spectra. Such computational efficiency in batch processing is critical for applications like real-time monitoring by Raman spectroscopy. In Table 6 the total computational times that ALS, Goldindec, and ISREA respectively take for 1000 simulated spectra and 50 spectra of dialysate samples are presented. As the table shows, both ALS and ISREA are clearly efficient in computation and ideal for batch processing.

TABLE 6
Computational time (in seconds) of ALS,
Goldindec and ISREA when
processing batches of simulated spectra
and dialysate spectra.

| | 1000 simulated spectra | 50 dialysis spectra |
|---|---|---|
| ALS | 3.16 | 0.16 |
| Goldindec | 885.36 | 38.54 |
| ISREA | 24.13 | 8.76 |

Cohort Description for the Hemodialysis Study

The study cohort consisted of 10 chronic in-center hemodialysis patients. Mean age in the cohort was 69.9 years (median 72.5), 50% of participants were female, and 20% were black or African American and 80% were white or Caucasian American. Comorbid conditions for the group included coronary artery disease (50%), hypertension (100%), type 2 diabetes mellitus (40%), and congestive heart failure (20%). None of the subjects had active cancer. Eighty percent of the patients dialyzed using an arterio-venous fistula, compared to 20% with a central venous catheter. Dialysis vintage, i.e., the duration of time between the first day of dialysis treatment and the day of kidney transplantation, varied from 8-87 months (mean: 33.7 months, median: 21.5 months). Average prescribed dialysis time was 200 minutes (median 202 minutes) and all patients had stable single pool Kt/V urea values greater than 1.2.

Sensitivity Analysis on ε for ISREA

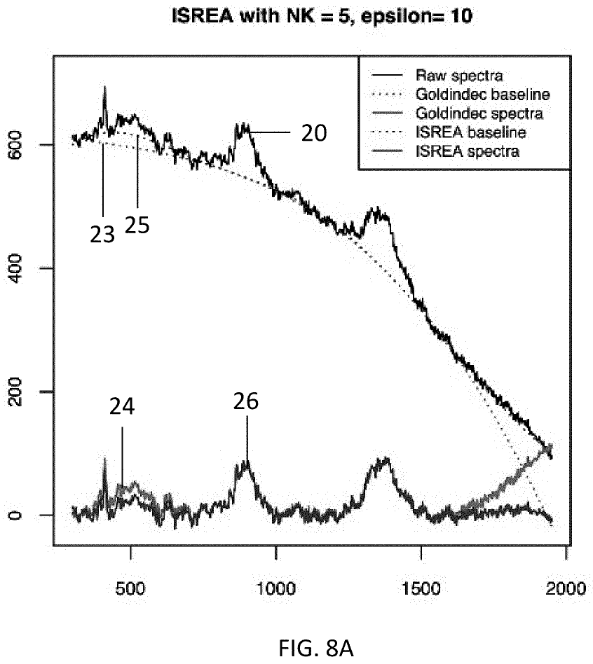

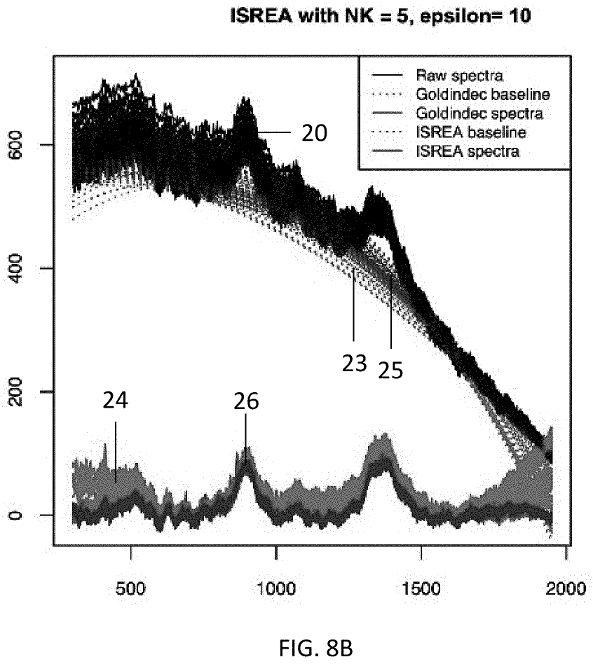

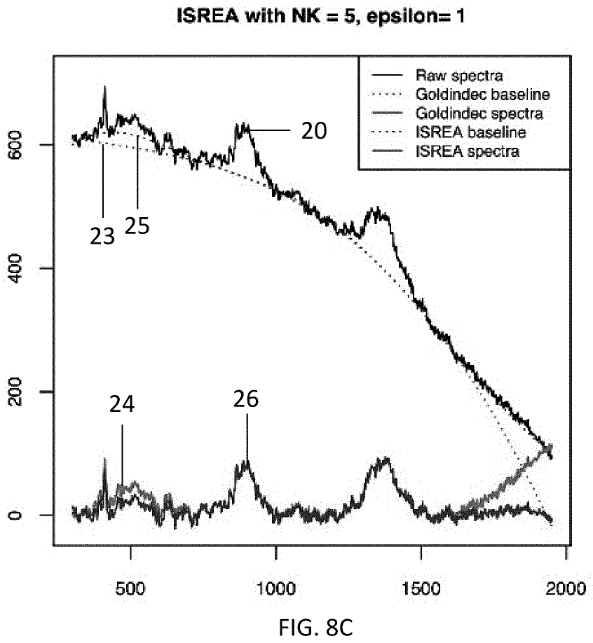

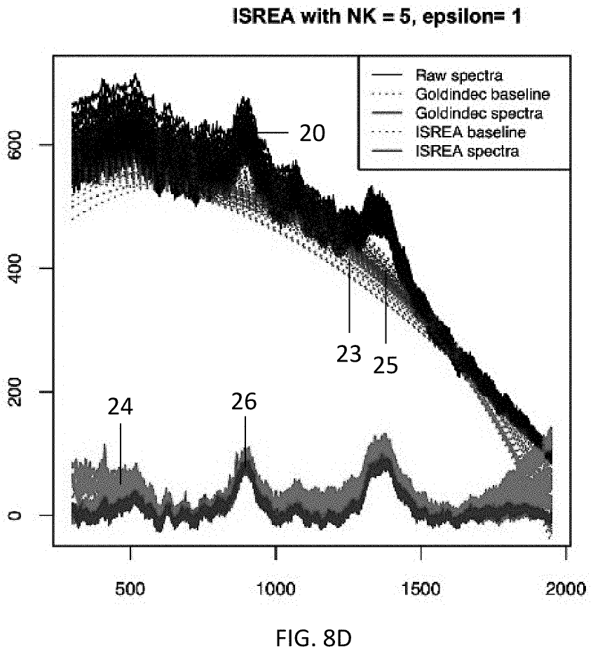

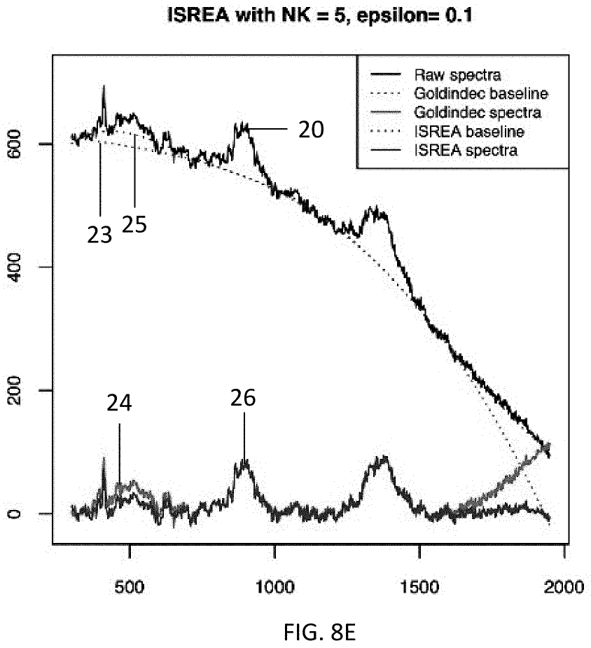

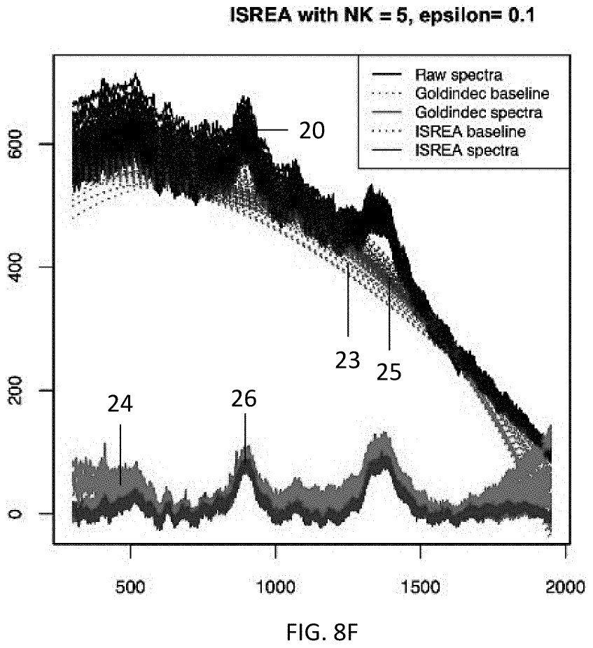

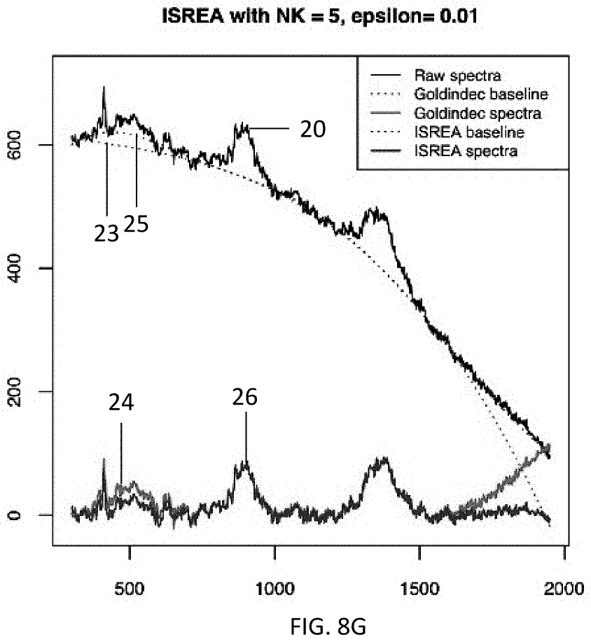

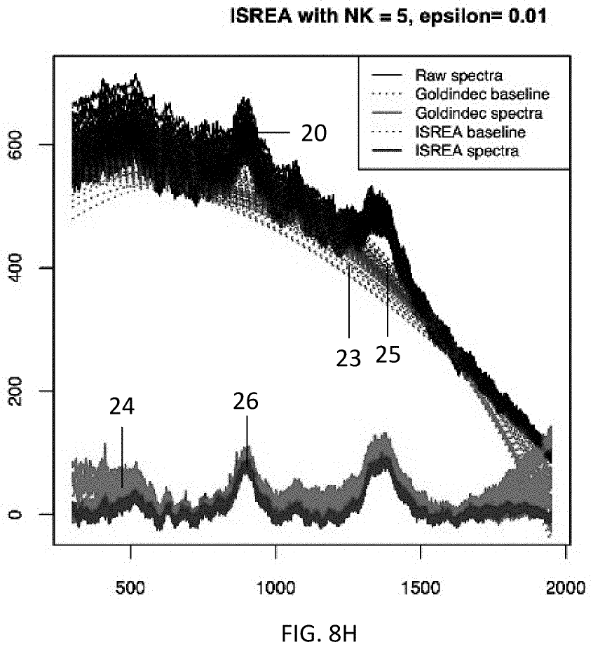

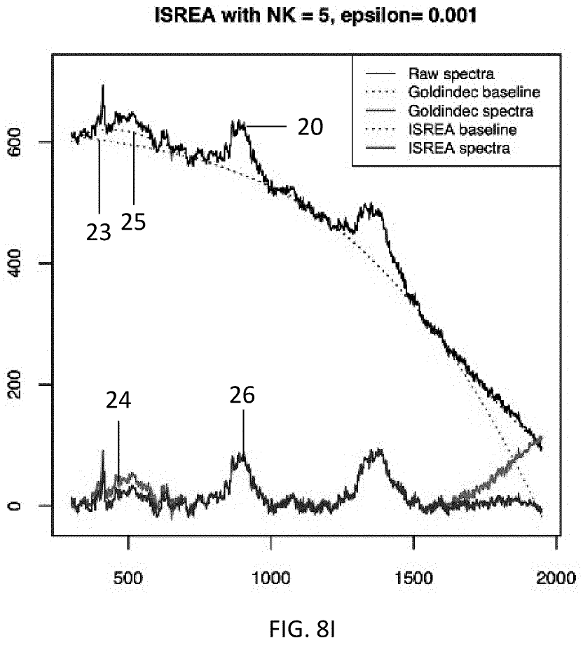

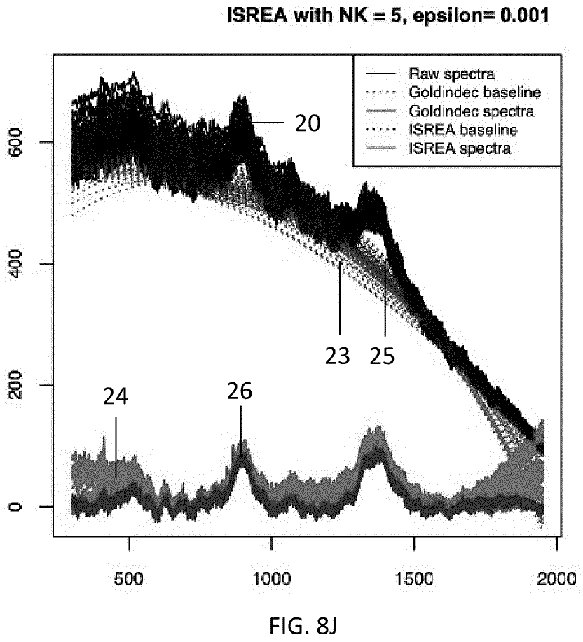

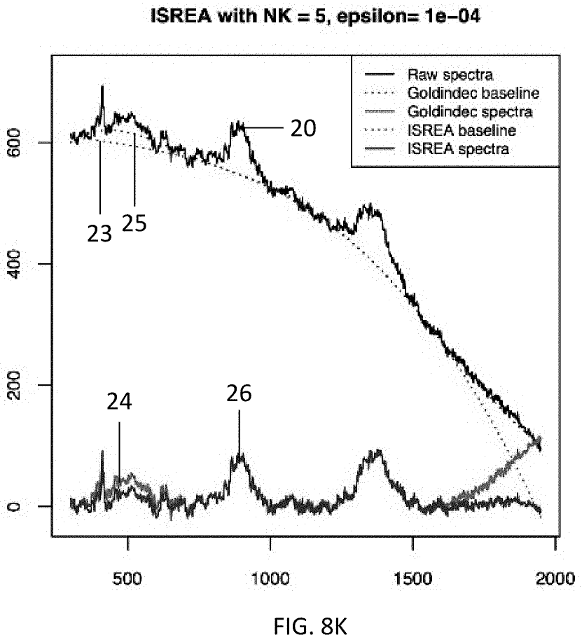

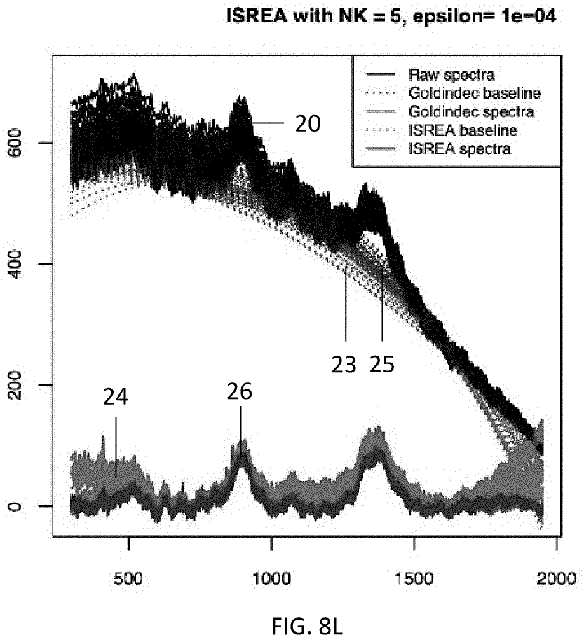

The ISREA algorithm stops its iterations when the change in the baseline estimates fall below E. We conducted numeric experiments to study the effects of different choices of ε on the performance of ISREA respectively in the scenarios of 1000 simulated spectra, six mineral spectra, and 50 dialysis spectra. Table 7 shows the percentiles of AC rates on 1000 simulated spectra. Table 8 contains the AC_rates for the six mineral spectra. For 50 dialysis spectra, since there are no true or expert-correction baselines, visual illustration is provided instead in FIGS. 8A-L. As illustrated, the ISREA is not sensitive to the choice of ε and a much smaller ε does not improve the performance significantly. Note that a smaller ε also means more computational time due to slower convergence. Accordingly, ε=10 appears to be sufficient for all experiments as well as converges reasonably fast.

TABLE 7
Percentiles of AC_rates for ISREA on 1000
simulated spectra at different ε

| ε | 0% | 5% | 25% | 50% | 75% | 95% | 100% | Mean |
|---|---|---|---|---|---|---|---|---|
| 10 | 0.6899 | 0.9340 | 0.9840 | 0.9934 | 0.9965 | 0.9985 | 0.9995 | 0.9836 |
| 1 | 0.6899 | 0.9350 | 0.9842 | 0.9935 | 0.9965 | 0.9985 | 0.9995 | 0.9838 |
| 0.1 | 0.6899 | 0.9354 | 0.9843 | 0.9935 | 0.9965 | 0.9985 | 0.9995 | 0.9839 |
| 0.01 | 0.6909 | 0.9355 | 0.9843 | 0.9935 | 0.9965 | 0.9985 | 0.9995 | 0.9839 |
| 0.001 | 0.6911 | 0.9355 | 0.9843 | 0.9935 | 0.9965 | 0.9985 | 0.9995 | 0.9839 |
| 0.0001 | 0.6911 | 0.9355 | 0.9843 | 0.9935 | 0.9965 | 0.9985 | 0.9995 | 0.9839 |

TABLE 8
AC_rates of ISREA on mineral spectra with different ε

| ε | Andersonite | Eastonite | Marialite | Parascholzite | Sugilite | Wadeite |
|---|---|---|---|---|---|---|
| 10 | 0.9861 | 0.9818 | 0.9952 | 0.9825 | 0.9870 | 0.8984 |
| 1 | 0.9868 | 0.9818 | 0.9951 | 0.9824 | 0.9872 | 0.8951 |
| 0.1 | 0.9871 | 0.9818 | 0.9951 | 0.9824 | 0.9873 | 0.8942 |
| 0.01 | 0.9872 | 0.9818 | 0.9951 | 0.9824 | 0.9875 | 0.8938 |
| 0.001 | 0.9872 | 0.9818 | 0.9951 | 0.9824 | 0.9875 | 0.8937 |
| 0.0001 | 0.9872 | 0.9818 | 0.9951 | 0.9824 | 0.9875 | 0.8937 |

Motivated from the success of asymmetric loss functions, ISREA is a simple and fast baseline correction procedure that mimics the threshold-free asymmetric square-root loss and well preserves all the meaningful Raman peaks. Its computational efficiency is a natural by-product deriving from the elimination of the threshold selection and non-convex optimization required in existing asymmetric loss methods. Numerical experiments have demonstrated that ISREA has excellent and consistent performance on a wide variety of spectra, including mineral spectra that consist of sharp or low but sparse peaks and spectra of complicated compounds like used dialysate that contain many meaningful peaks over the whole spectral domain. Although ISREA is implemented in R, it can be easily translated to other common languages like Python, MATLAB, and others to facilitate automation and processing of large Raman spectral datasets.

The comparisons of ISREA with the ALS and Goldindec procedures indicate that both ISREA and ALS can perform better baseline correction than the Goldindec. Against each other, ISREA and ALS each have slight advantages from different aspects. Additionally, other baseline correction procedures can be used in combination with ISREA and/or ALS, such as EMSC which produces spectra similar to the chosen reference spectrum.

The present invention has been described with reference to particular embodiments having various features. In light of the disclosure provided above, it will be apparent to those skilled in the art that various modifications and variations can be made in the practice of the present invention without departing from the scope or spirit of the invention. One skilled in the art will recognize that the disclosed features may be used singularly, in any combination, or omitted based on the requirements and specifications of a given application or design. When an embodiment refers to “comprising” certain features, it is to be understood that the embodiments can alternatively “consist of” or “consist essentially of” any one or more of the features. Other embodiments of the invention will be apparent to those skilled in the art from consideration of the specification and practice of the invention.

It is noted in particular that where a range of values is provided in this specification, each value between the upper and lower limits of that range is also specifically disclosed. The upper and lower limits of these smaller ranges may independently be included or excluded in the range as well. The singular forms “a,” “an,” and “the” include plural referents unless the context clearly dictates otherwise. It is intended that the specification and examples be considered as exemplary in nature and that variations that do not depart from the essence of the invention fall within the scope of the invention. Further, all of the references cited in this disclosure are each individually incorporated by reference herein in their entireties and as such are intended to provide an efficient way of supplementing the enabling disclosure of this invention as well as provide background detailing the level of ordinary skill in the art.

### REFERENCES (INCORPORATED HEREIN BY REFERENCE IN THEIR ENTIRETIES)

A. Kudelski. “Analytical applications of Raman spectroscopy”. Talanta. 2008. 76(1): 1-8.
R. S. Das, Y. K. Agrawal. “Raman spectroscopy: Recent advancements, techniques and applications”. Vib. Spectrosc. 2011. 57(2): 163-176.
N. Wang, H. Cao, L. Wang, F. Ren, Q. Zeng, X. Xu, et al. “Recent advances in spontaneous Raman spectroscopic imaging: Instrumentation and applications”. Curr. Med. Chem. 2019. 26: 1.
J. Depciuch, E. Kaznowska, I. Zawlik, R. Wojnarowska, M. Cholewa, P. Heraud, et al. “Application of Raman Spectroscopy and Infrared Spectroscopy in the Identification of Breast Cancer”. Appl. Spectrosc. 2016. 70(2): 251-263.
A. K. Fisher, W. F. Carswell, A. I. M. Athamneh, M. C. Sullivan, J. L. Robertson, D. R. Bevan, et al. “The Rametrix™ LITE Toolbox v1.0 for MATHLAB®”. J. Raman Spectrosc. 2018. 49(5): 885-896.
R. S. Senger, V. Kavuru, M. Sullivan, A. Gouldin, S. Lundgren, K. Merrifield, et al. “Spectral characteristics of urine specimens from healthy human volunteers analyzed using Raman chemometric urinalysis (Rametrix)”. PLoS One. 2019. 14(9): e0222115.
R. S. Senger, J. L. Robertson. “The Rametrix™ PRO Toolbox v1.0 for MATLAB®”. PeerJ. 2020. 8: e8179.
R. S. Senger, M. Sullivan, A. Gouldin, S. Lundgren, K. Merrifield, C. Steen, et al. “Spectral characteristics of urine from patients with end-stage kidney disease analyzed using Raman Chemometric Urinalysis (Rametrix)”. PLoS One. 2020. 15(1): e0227281.
R. Gautam, S. Vanga, F. Ariese, S. Umapathy. “Review of multidimensional data processing approaches for Raman and infrared spectroscopy”. EPJ Techn. Instrum. 2015. 2(1): 8.
P. A. Mosier-Boss, S. H. Lieberman, R. Newbery. “Fluorescence rejection in Raman spectroscopy by shifted-spectra, edge detection, and FFT filtering techniques”. Appl. Spectrosc. 1995. 49: 630-638.
J. Zhao, H. Lui, D. I. McLean, H. Zeng. “Automated autofluorescence background subtraction algorithm for biomedical Raman spectroscopy”. Appl. Spectrosc. 2007. 61(11): 1225-1232.
A. Mahadevan-Jansen, R. R. Richards-Kortum. “Raman spectroscopy for the detection of cancers and precancers”. J. Biomed. Opt. 1996. 1(1): 31-70.
C. A. Lieber, A. Mahadevan-Jansen. “Automated Method for Subtraction of Fluorescence from Biological Raman Spectra”. Appl. Spectrosc. 2003. 57: 1363-1367.
C. M. Stellman, K. S. Booksh, M. L. Myrick. “Multivariate Raman Imaging of Simulated and “Real World” Glass-Reinforced Composites”. Appl. Spectrosc. 1996. 50: 552-557.
V. Shusterman, S. I. Shah, A. Beigel, K. P. Anderson. “Enhancing the precision of ECG baseline correction: selective filtering and removal of residual error”. Comput. Biomed. Res. 2000. 33(2): 144-160.
G. Schulze, A. Jirasek, M. M. Yu, A. Lim, R. F. Turner, M. W. Blades. “Investigation of selected baseline removal techniques as candidates for automated implementation”. Appl. Spectrosc. 2005. 59(5): 545-574.
T. T. Cai, D. Zhang, D. Ben-Amotz. “Enhanced Chemical Classification of Raman Images Using Multiresolution Wavelet Transformation”. Appl. Spectrosc. 2001. 55(9): 1124-1130.
D. Chen, Z. Chen, E. Grant. “Adaptive wavelet transform suppresses background and noise for quantitative analysis by Raman spectrometry”. Anal. Bioanal. Chem. 2011. 400(2): 625-634.
J. Li, B. Yu, H. Fischer. “Wavelet transform based on the optimal wavelet pairs for tunable diode laser absorption spectroscopy signal processing”. Appl. Spectrosc. 2015. 69(4): 496-506.
D. Zhang, D. Ben-Amotz. “Enhanced Chemical Classification of Raman Images in the Presence of Strong Fluorescence Interference”. Appl. Spectrosc. OSA, 2000. 54(9): 1379-1383.
Y. Cai, C. Yang, D. Xu, W. Gui. “Baseline correction for Raman spectra using penalized spline smoothing based on vector transformation”. Anal. Methods. 2018. 10(28): 3525-3533.
V. Mazet, C. Carteret, D. Brie, J. Idier, B. Humbert. “Background removal from spectra by designing and minimising a non-quadratic cost function”. Chemom. Intell. Lab. Syst. 2005. 76(2): 121-133.
J. R. Beattie, J. J. McGarvey. “Estimation of signal backgrounds on multivariate loadings improves model generation in face of complex variation in backgrounds and constituents”. J of Raman Spectrosc. 2013. 44(2): 329-338. 10.1002/jrs.4178.
H. Martens, E. Stark. “Extended multiplicative signal correction and spectral interference subtraction: new preprocessing methods for near infrared spectroscopy.” J. Pharm. Biomed. Anal. 1991. 9 8: 625-635.
K. Liland, A. Kohler, N. Afseth. “Model-based pre-processing in Raman spectroscopy of biological samples”. J. Raman Spectrosc. 2016. 47(6): 643-650.
M. Scholtes-Timmerman, H. Willemse-Erix, T. B. Schut, A. van Belkum, G. Puppels, K. Maquelin. “A novel approach to correct variations in Raman spectra due to photo-bleachable cellular components”. Analyst. 2009. 134(2): 387-393.
P. Candeloro, E. Grande, R. Raimondo, D. D. Mascolo, F. Gentile, M. L. Coluccio, et al. “Raman database of amino acids solutions: A critical study of extended multiplicative signal correction”. Analyst. 2013. 138(24): 7331-7340.
N. K. Afseth, A. Kohler. “Extended multiplicative signal correction in vibrational spectroscopy, a tutorial”. Chemometrics and Intelligent Laboratory Systems. 2012. 117: 92-99. 10.1016/j.chemolab.2012.03.004.
J. Liu, J. Sun, X. Huang, G. Li, B. Liu. “Goldindec: A Novel Algorithm for Raman Spectrum Baseline Correction”. Appl. Spectrosc. 2015. 69(7): 834-842.
P. H. C. Eilers. “A perfect smoother”. Anal. Chem. 2003. 75(14): 3631-3636.
K. Liland, T. Almøy, B. Mevik. “Optimal Choice of Baseline Correction for Multivariate Calibration of Spectra”. Appl. Spectrosc. 2010. 64: 1007-1016.
J. Peng, S. Peng, J. A, J. Jiping Wei, C. Changwen Li, J. Jie Tan. “Asymmetric least squares for multiple spectra baseline correction”. Anal. Chim. Acta. 2010. 683(1): 63-68.
S. He, W. Zhang, L. Liu, Y. Huang, J. He, W. Xie, et al. “Baseline correction for Raman spectra using an improved asymmetric least squares method”. Anal. Methods. The Royal Society of Chemistry, 2014. 6(12): 4402-4407.
H. F. M. Boelens, P. H. C. Eilers. Baseline correction with asymmetric least squares smoothing. Leiden University Medical Centre report. 2005.
B. Lafuente, R. T. Downs, Y. H, S. N. “The power of databases: the RRUFF project”. In: T. Armbruster, R. M. Danisi, editors. Highlights in Mineralogical Crystallography. W. De Gruyter, Berlin, Germany, 2015. Pp. 1-30.
Xu, Y, et. al. (2020): Xu, Y, Du, P, Senger, R, Robertson, J, Pirkle, J “ISREA: An efficient peak-preserving baseline correction algorithm for Raman spectra,” First Published Oct. 8, 2020 https://doi.org/10.1177/0003702820955245.

1. A method of identifying and/or quantifying a condition of a subject comprising:
obtaining a Raman spectrum from a sample from a subject;
obtaining a transformed, baseline-corrected Raman spectrum by baselining and transforming the Raman spectrum by:
(a) obtaining a baseline estimate on the Raman spectrum by fitting smoothing splines to the Raman spectrum;
(b) determining a difference in intensity value at one or more wavenumber of the Raman spectrum as compared with a corresponding wavenumber of the baseline estimate;
(c) obtaining an adjusted baseline estimate by adjusting the intensity values of the baseline estimate where a positive difference is determined, and where there is a zero or negative difference determined, then the intensity value of the initial baseline estimate remains the same;
(d) iterating by repeating (a)-(c) on the adjusted baseline estimates; and
(e) obtaining the transformed, baseline-corrected Raman spectrum by repeating (d) until a desired deviation between two consecutive adjusted baseline estimates is reached;
optionally performing node optimization on the baseline-corrected Raman spectrum;
analyzing the baseline-corrected Raman spectrum to detect the presence of and/or quantify a condition of the subject by analyzing peak position, height and/or area under the curve of one or more peaks of interest.

2. The method of claim 1, wherein the analyzing comprises one or more of principal component analysis (PCA), discriminant analysis of principal components (DAPC), partial least squares (PLS), and/or artificial neural networks (NN) to detect and/or quantify the condition.

3. The method of claim 2, wherein the analyzing comprises determining whether the baseline-corrected Raman spectrum is classified as being (a) from a subject who has the specified condition or (b) from a subject who does not have the specified condition and is performed in a manner such that it is determined that baseline-corrected Raman spectrum fits closer mathematically to one or the other statistically significant groups (a) or (b).

4. The method of claim 1, wherein the condition of the subject is any one or more of Bladder cancer (all types, grades, and stages); Acute cystitis (all types, grades, stages, and etiologies, including infectious and non-infectious etiologies); Chronic cystitis (all types, grades, stages, and etiologies, including infectious and non-infectious etiologies); Schistosomiasis; Kidney cancer (all types, grades and stages); Prostate cancer (all types, grades, and stages); Prostatitis (acute and chronic); Cervical cancer (all types, grades, and stages); Uterine cancer (all types, grades, and stages); Ovarian cancer (all types, grades, and stages); Cancer of the adrenal gland (all types, grades, and stages); Cushing's disease and Cushing's syndrome; Multiple myeloma with Bence-Jones proteinuria (all stages and grades); Acute kidney injury (all types and etiologies); Acute kidney failure (all types and etiologies); Chronic kidney failure (all types, stages, and etiologies); Acute glomerulonephritis (all types and etiologies); Chronic glomerulonephritis (all types and etiologies); Focal and diffuse segmental glomerulosclerosis (all stages, grades, and etiologies, including hypertension); Membranous nephropathy (all stages, grades, and etiologies); Membranoproliferative glomerulonephritis (all stages, grades, and etiologies, including systemic lupus erythematosus); Hemolytic uremic syndrome; IgA nephropathy (all stages, grades, and etiologies); Minimal change nephropathy (all stages, grades, and etiologies); Congenital nephropathy (all stages, grades, and etiologies); Diabetes; Diabetic nephropathy; Protein-losing nephropathy and nephrotic syndrome (all stages, grades, and etiologies); Acute pyelonephritis (all stages, grades, and etiologies); Chronic pyelonephritis (all stages, grade, and etiologies); Lyme disease (all stages and clinical presentations); Atypical borreliosis; Myalgic Encephalomyelitis/Chronic Fatigue Syndrome (ME/CFS) (all types, stages, and etiologies); Systemic mold allergy/toxicity; Hemobartonellosis; SARS-CoV-1 (Severe Acute Respiratory Syndrome Coronavirus Disease); SARS-CoV-2 (COVID-19 Disease); and MERS-CoV-2 (Middle Eastern Respiratory Syndrome Disease).

5. The method of claim 1, wherein the presence of the condition of the subject is made visible in the baseline-corrected Raman spectrum by emphasizing one or more of the peaks of interest and/or minimizing other peak(s).

6. A method of producing a transformed, baseline-corrected Raman spectrum comprising:
applying an iterative fitting procedure to a Raman spectrum to adjust for peak invasion;
in each iteration, prediction errors are adjusted down through a root transformation and added back to fitted baseline intensities to form a new set of intensities;
applying smoothing splines to the new set of intensities to obtain a new baseline estimate, based on which a new set of prediction errors are calculated; and
repeating the fitting procedure and stopping when errors fall below a desired level, such that a transformed, baseline-corrected Raman spectrum is obtained.

7. The method of claim 6, wherein the fitting procedure comprises adjusting intensity values of the fitted baseline intensities and/or the new set of intensities where a positive difference is determined, and where there is a zero or negative difference determined, then the intensity value remains the same.

8. A method of producing a transformed, baseline-corrected Raman spectrum comprising:
(a) obtaining a baseline estimate on a Raman spectrum by fitting smoothing splines to the Raman spectrum;
(b) determining a difference in intensity value at one or more wavenumber of the Raman spectrum as compared with a corresponding wavenumber of the baseline estimate;
(c) obtaining an adjusted baseline estimate by adjusting the intensity values of the baseline estimate where a positive difference is determined, and where there is a zero or negative difference determined, then the intensity value of the initial baseline estimate remains the same;
(d) iterating by repeating (a)-(c) on the adjusted baseline estimates; and
(e) obtaining a transformed, baseline-corrected Raman spectrum by repeating (d) until a desired deviation between two consecutive adjusted baseline estimates is reached.

9. The method of claim 8, wherein the adjusting of the intensity values is performed according to:
$${y}_{i}^{( new )} = {\hat{m}}_{i} + \sqrt[4]{{δ}_{i}} ,$$
wherein:
yᵢ^{(new)} is an adjusted intensity value;
the difference in intensity value δᵢ is defined as: δᵢ=yᵢ−\hat{m}ᵢ;
yᵢ is an intensity value of the Raman spectrum; and
\hat{m}ᵢ is an intensity value of the baseline estimate.

10. The method of claim 8, wherein the iterating (d) is repeated until the desired deviation falls below a selected convergence criterion ε.

11. The method of claim 10, wherein the convergence criterion c is a number in the range of from 0.0001 to 10.

12. The method of claim 8, wherein a number of knots for the smoothing spline is a number in the range of from 5 to 20.

13. The method of claim 8, wherein the Raman spectrum is from a fluid, tissue, gas, and/or solid.

14. The method of claim 13, wherein the Raman spectrum is from a dialysate or urine sample.

15. The method of claim 8, comprising:
(a) obtaining an initial smooth baseline estimate by fitting smoothing splines to a raw Raman spectrum, where δᵢ=yᵢ−\hat{m}ᵢ is the deviation of the smooth baseline, \hat{m}ᵢ, from an observed raw spectral intensity, yᵢ;
(b) adjusting intensities such that areas with zero or negative intensity deviations, δᵢ≤0, remain the same, while areas with positive intensity deviations, δᵢ>0, are updated as
$${y}_{i}^{( n e w )} = {\hat{m}}_{i} + \sqrt[4]{{δ}_{i}} ;$$
(c) feeding the new intensities, yᵢ^{(new)}, into the smoothing spline estimation again to get an updated baseline function estimate, \hat{ƒ}, and thus updated baseline intensity estimates \hat{m}ᵢ=\hat{ƒ}(i/n);
(d) repeating one or more of steps (a)-(c) until a difference between two consecutive fitted baselines is small.

16. The method of claim 15, wherein the Raman spectrum is modeled as:
y=m+a+ε,
where m_{n×1}=(m₁, . . . , mₙ)^{T}, a_{n×1}=(a₁, . . . , aₙ)^{T}, and ϵ_{n×1}=(ϵ₁, . . . , ϵₙ)^{T} are respectively vectors of unknown true baseline intensities, peak intensities, and random noises.

17. The method of claim 5, wherein the node optimization is performed by placing a selected number of nodes to highlight regions of the Raman spectrum in which one or more compound(s) of interest appears and/or to highlight one or more regions where the Raman spectrum and the baseline do not overlap.

18. The method of claim 1, wherein the node optimization is performed such that a selected number of nodes are placed to highlight one or more areas of interest, and/or glucose, in a urine spectrum to detect diabetes.

19. The method of claim 1, wherein the node optimization is performed such that a selected number of nodes are placed to highlight one or more areas of interest in a patient spectrum related to and to detect hypertension.

20. The method of claim 8, wherein knots for the smoothing spline are placed to highlight regions of the Raman spectrum in which one or more compound(s) of interest appears and/or are placed to highlight one or more regions where the Raman spectrum and the baseline do not overlap.
